SEQUENCE LISTING (26 sequences)

SEQ ID NO: 1 - RNA, 25 nt, Artificial Sequence
  synthetic
acccugaaguucaucugcaccaccg

SEQ ID NO: 2 - RNA, 25 nt, Artificial Sequence
  synthetic
acccugaaguucaucugcaccaccg

SEQ ID NO: 3 - DNA, 19 bp, Artificial Sequence
  synthetic
cagatgaacttcagggtca

SEQ ID NO: 4 - DNA, 21 bp, Artificial Sequence
  synthetic
tgcagatgaacttcagggtca

SEQ ID NO: 5 - DNA, 23 bp, Artificial Sequence
  synthetic
ggtgcagatgaacttcagggtca

SEQ ID NO: 6 - DNA, 25 bp, Artificial Sequence
  synthetic
gtggtgcagatgaacttcagggtca

SEQ ID NO: 7 - RNA, 27 nt, Artificial Sequence
  synthetic
cgguggugcagaugaacuucaggguca

SEQ ID NO: 8 - RNA, 27 nt, Artificial Sequence
  synthetic
cgguggugcagaugaacuucaggguca

SEQ ID NO: 9 - DNA, 19 bp, Artificial Sequence
  synthetic
tgaccctgaagttcatctg

SEQ ID NO: 10 - DNA, 21 bp, Artificial Sequence
  synthetic
tgaccctgaagttcatctgca

SEQ ID NO: 11 - DNA, 23 bp, Artificial Sequence
  synthetic
tgaccctgaagttcatctgcacc

SEQ ID NO: 12 - DNA, 25 bp, Artificial Sequence
  synthetic
tgaccctgaagttcatctgcaccac

SEQ ID NO: 13 - RNA, 25 nt, Artificial Sequence
  synthetic
gaagaaaugaugacagcauuucagg

SEQ ID NO: 14 - DNA, 19 bp, Artificial Sequence
  synthetic
gctgtcatcatttcttctt

SEQ ID NO: 15 - RNA, 27 nt, Artificial Sequence
  synthetic
ccugaaaugcugucaucauuucuucuu

SEQ ID NO: 16 - DNA, 19 bp, Artificial Sequence
  synthetic
aagaagaaatgatgacagc

SEQ ID NO: 17 - RNA, 25 nt, Artificial Sequence
  synthetic
ggagagagaugggugcgaguucguc

SEQ ID NO: 18 - DNA, 19 bp, Artificial Sequence
  synthetic
cgcacccatctctctcctt

SEQ ID NO: 19 - RNA, 27 nt, Artificial Sequence
  synthetic
gacggacucgcacccaucucucuccuu

SEQ ID NO: 20 - DNA, 19 bp, Artificial Sequence
  synthetic
aaggagagagatgggtgcg

SEQ ID NO: 21 - RNA, 73 nt, Artificial Sequence
  synthetic
gggaaccguccacugguucccgcuacgagagccugccucguagcuucgguggugcagaugaacuucaggguca

SEQ ID NO: 22 - RNA, 73 nt, Artificial Sequence
  synthetic
gggaaccgcaggcugguucccgcuacgagagaacgccucguagcuucgguggugcagaugaacuucaggguca

SEQ ID NO: 23 - RNA, 73 nt, Artificial Sequence
  synthetic
gggaaccgcguucugguucccgcuacgagacgucuccucguagcuucgguggugcagaugaacuucaggguca

SEQ ID NO: 24 - RNA, 73 nt, Artificial Sequence
  synthetic
gggaaccgagacgugguucccgcuacgagucguggucucguagcuucgguggugcagaugaacuucaggguca

SEQ ID NO: 25 - RNA, 73 nt, Artificial Sequence
  synthetic
gggaaccaccacgagguucccgcuacgagaaccauccucguagcuucgguggugcagaugaacuucaggguca

SEQ ID NO: 26 - RNA, 73 nt, Artificial Sequence
  synthetic
gggaaccgaugguugguucccgcuacgagaguggaccucguagcuucgguggugcagaugaacuucaggguca

(19) United States
(12) Patent Application Publication
(10) Pub. No.: US 2021/0162066 A1
(43) Pub. Date: Jun. 3, 2021

(54) RNA/DNA HYBRID NANOPARTICLES MODIFIED WITH SINGLE STRANDED RNA TOEHOLDS AND USES THEREOF

(71) Applicant: The United States of America, as represented by the Secretary, Department of Health and Human Servic, Bethesda, MD (US)

(72) Inventors: Bruce Allen Shapiro, Gaithersburg, MD (US); Kirill Andreevich Afonin, Charlotte, NC (US); Mathias D. Viard, Frederick, MD (US); Eckart H.U. Bindewald, Frederick, MD (US); Lorena Parlea, Frederick, MD (US)

(73) Assignee: The United States of America,as represented by the Secretary,Department of Health and Human Services, Bethesda, MD (US)

(21) Appl. No.: 17/102,786

(22) Filed: Nov. 24, 2020

Related U.S. Application Data
(62) Division of application No. 16/076,878, filed on Aug. 9, 2018.
(60) Provisional application No. 62/294,848, filed on Feb. 12, 2016.

(51) Int. Cl. A61K 47/69; C12N 15/87; C12N 15/11; A61K 47/54; C12N 15/113

(52) U.S. Cl. CPC A61K 47/6929; C12N 15/87; C12N 15/111; A61K 47/6925; B82Y 5/00; C12N 15/113; C12N 2310/14; C12N 2320/50; C12N 2330/50; A61K 47/555

(57) ABSTRACT

The invention discloses the use of single-stranded RNA toeholds of different lengths to promote the re-association of various RNA-DNA hybrids, which results in activation of multiple split functionalities inside human cells. Previously designed RNA/DNA nanoparticles employed single-stranded DNA toeholds to initiate re-association. The use of RNA toeholds is advantageous because of the simpler design rules, the shorter toeholds, and the smaller size of the resulting nanoparticles compared to the same hybrid nanoparticles with single-stranded DNA toeholds. Moreover, the co-transcriptional assemblies result in higher yields for hybrid nanoparticles with ssRNA toeholds.

### CROSS-REFERENCE TO RELATED APPLICATIONS

This patent application is a divisional application of U.S. patent application Ser. No. 16/076,878, filed on Aug. 9, 2018, which is a U.S. national phase application of International Patent Application No. PCT/US2017/017661, filed on Feb. 13, 2017, which claims the benefit of U.S. Provisional Patent Application No. 62/294,848, filed on Feb. 12, 2016, each of which are incorporated by reference in their entirety herein.

### STATEMENT REGARDING FEDERALLY SPONSORED RESEARCH OR DEVELOPMENT

This invention was made with Government support under project numbers 1ZIABC008382-34 and 1ZIABC011061-10 by the National Institutes of Health, National Cancer Institute. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,650 Byte ASCII (Text) file named “751183_ST25.txt,” dated Nov. 23, 2020.

### INCORPORATION BY REFERENCE

This application generally relates to the design, preparation, and therapeutic/diagnostic application of specialized RNA/DNA nanoparticles which comprise single-stranded RNA (“ssRNA”) toehold sequences that improve their structure and function as described here. In general and where applicable, this specification incorporates by reference in their entireties Applicant's prior patent applications relating to RNA/DNA nanoparticles, which include PCT/US2007/013027 (WO2008/039254) (“RNA Nanoparticles and Nanotubes”), filed May 31, 2007, PCT/US2010/038818 (WO 2010/148085) (“RNA Nanoparticles and Nanotubes”), filed Jun. 16, 2010, PCT/US2012/065932 (WO 2013/075132)(“Therapeutic RNA Switches), filed Nov. 19, 2012, PCT/US2012/065945 (WO 2013/075140) (“Auto-Recognizing Therapeutic RNA/DNA Chimeric Nanoparticles”), filed Nov. 19, 2012, PCTUS2013/058492 (WO 2014/039809) (“Co-Transcriptional Assembly of Modified RNA Nanoparticles”), filed Sep. 6, 2013, PCT/US2014/056007 (WO 2015/042101) (“Multifunctional RNA Nanoparticles and Methods of Use”), filed Sep. 17, 2014, and PCT/US2015/029553 (WO 2015/171827) (“Triggering RNA Interference with RNA-DNA and DNA-RNA Nanoparticles”), filed May 6, 2015.

In addition, this application incorporates by reference all of Applicant's prior published scientific journal articles relating to RNA/DNA nanoparticles and the above-indicated prior applications, which include, but is not limited to:
1: Halman J R, Satterwhite E, Roark B, Chandler M, Viard M, Ivanina A, Bindewald E, Kasprzak W K, Panigaj M, Bui M N, Lu J S, Miller J, Khisamutdinov E F, Shapiro B A, Dobrovolskaia M A, Afonin K A. Functionally-interdependent shape-switching nanoparticles with controllable properties. Nucleic Acids Res. 2017 Jan. 20. pii: gkx008. doi: 10.1093/nar/gkx008. [Epub ahead of print] PubMed PMID: 28108656.
2: Parlea L, Puri A, Kasprzak W, Bindewald E, Zakrevsky P, Satterwhite E, Joseph K, Afonin K A, Shapiro B A. Cellular Delivery of RNA Nanoparticles. ACS Comb Sci. 2016 Sep. 12; 18(9):527-47. doi: 10.1021/acscombsci.6b00073. PubMed PMID: 27509068.
3: Parlea L, Bindewald E, Sharan R, Bartlett N, Moriarty D, Oliver J, Afonin K A, Shapiro B A. Ring Catalog: A resource for designing self-assembling RNA nanostructures. Methods. 2016 Jul. 1; 103:128-37. doi: 10.1016/j.ymeth.2016.04.016. PubMed PMID: 27090005.
4: Afonin K A, Viard M, Tedbury P, Bindewald E, Parlea L, Howington M, Valdman M, Johns-Boehme A, Brainerd C, Freed E O, Shapiro B A. The Use of Minimal RNA Toeholds to Trigger the Activation of Multiple Functionalities. Nano Lett. 2016 Mar. 9; 16(3):1746-53. doi: 10.1021/acs.nanolett.5b04676. PubMed PMID: 26926382.
5: Afonin K A, Viard M, Kagiampakis I, Case C L, Dobrovolskaia M A, Hofmann J, Vrzak A, Kireeva M, Kasprzak W K, KewalRamani V N, Shapiro B A. Triggering of RNA interference with RNA-RNA, RNA-DNA, and DNA-RNA nanoparticles. ACS Nano. 2015 Jan. 27; 9(1):251-9. doi: 10.1021/nn504508s. PubMed PMID: 25521794; PubMed Central PMCID: PMC4310632.
6: Afonin K A, Viard M, Koyfman A Y, Martins A N, Kasprzak W K, Panigaj M, Desai R, Santhanam A, Grabow W W, Jaeger L, Heldman E, Reiser J, Chiu W, Freed E O, Shapiro B A. Multifunctional RNA nanoparticles. Nano Lett. 2014 Oct. 8; 14(10):5662-71. doi: 10.1021/n1502385k. PubMed PMID: 25267559; PubMed Central PMCID: PMC4189619.
7: Afonin K A, Kasprzak W K, Bindewald E, Kireeva M, Viard M, Kashlev M, Shapiro B A. In silico design and enzymatic synthesis of functional RNA nanoparticles. Acc Chem Res. 2014 Jun. 17; 47(6):1731-41. doi: 10.1021/ar400329z. PubMed PMID: 24758371; PubMed Central PMCID: PMC4066900.
8: Afonin K A, Desai R, Viard M, Kireeva M L, Bindewald E, Case C L, Maciag A E, Kasprzak W K, Kim T, Sappe A, Stepler M, Kewalramani V N, Kashlev M, Blumenthal R, Shapiro B A. Co-transcriptional production of RNA-DNA hybrids for simultaneous release of multiple split functionalities. Nucleic Acids Res. 2014 February; 42(3):2085-97. doi: 10.1093/nar/gkt1001. PubMed PMID: 24194608; PubMed Central PMCID: PMC3919563.
9: Afonin K A, Viard M, Martins A N, Lockett S J, Maciag A E, Freed E O, Heldman E, Jaeger L, Blumenthal R, Shapiro B A. Activation of different split functionalities on re-association of RNA-DNA hybrids. Nat Nanotechnol. 2013 April; 8(4):296-304. doi: 10.1038/nnano.2013.44. PubMed PMID: 23542902; PubMed Central PMCID: PMC3618561.
10: Afonin K A, Kireeva M, Grabow W W, Kashlev M, Jaeger L, Shapiro B A. Co-transcriptional assembly of chemically modified RNA nanoparticles functionalized with siRNAs. Nano Lett. 2012 Oct. 10; 12(10):5192-5. doi: 10.1021/n302302e. PubMed PMID: 23016824; PubMed Central PMCID: PMC3498980.
11: Afonin K A, Grabow W W, Walker F M, Bindewald E, Dobrovolskaia M A, Shapiro B A, Jaeger L. Design and self-assembly of siRNA-functionalized RNA nanoparticles for use in automated nanomedicine. Nat Protoc. 2011 Dec. 1; 6(12):2022-34. Doi 10.1038/nprot.2011.418. PubMed PMID: 22134126; PubMed Central PMCID: PMC3498981.
12: Shapiro B A, Bindewald E, Kasprzak W, Yingling Y. Protocols for the in silico design of RNA nanostructures. Methods Mol Biol. 2008; 474:93-115. doi: 10.1007/978-1-59745-480-3_7. Review. PubMed PMID: 19031063.
13: Bindewald E, Grunewald C, Boyle B, O'Connor M, Shapiro B A. Computational strategies for the automated design of RNA nanoscale structures from building blocks using NanoTiler. J Mol Graph Model. 2008 October; 27(3):299-308. Doi: 10.1016/j.jmgm.2008.05.004. PubMed PMID: 18838281; PubMed Central PMCID: PMC3744370.
14: Yingling Y G, Shapiro B A. Computational design of an RNA hexagonal nanoring and an RNA nanotube. Nano Lett. 2007 August; 7(8):2328-34. PubMed PMID: 17616164.

### BACKGROUND OF THE INVENTION

The development of RNA-based nanostructures and their use in a variety of applications, including RNA interference (RNAi) and drug delivery, represents an emerging field of science, technology, and biomedicine often referred to as RNA nanobiology. RNA itself represents a relatively new molecular material for the development of such nanostructures. RNA is a dynamic material because of its natural functionalities, its ability to fold into complex structures, and its capacity to self-assemble. Despite much progress, RNA nanobiology is relatively young and continual improvements are needed in order to fully harness the significant potential of RNA nanostructures in the biomedical arts. In one aspect, RNA nanobiology provides new possibilities for the diagnosis and treatment of various diseases, such as cancer and viral infections. In particular, RNA nanoparticles are ideal drug delivery devices due to their novel properties and functions and ability to operate at the same scale as biological entities. Nanoparticles, because of their small size, can penetrate through smaller capillaries and are taken up by cells, which allow efficient drug accumulation at the target sites (Panyam J et al., Fluorescence and electron microscopy probes for cellular and tissue uptake of poly (D, L-lactide-co-glycolide) nanoparticles, Int J Pharm. 262:1-11, 2003).

There are several issues that are important for efficient design and drug delivery by nanoparticles, including the efficient attachment of drugs and vectors, controlled drug release, size, toxicity, biodegradability, and activity of the nanoparticle. Moreover, for successful design one needs to understand and control the intermolecular associations, based on natural favorability of interactions and various physical components. Targeted delivery of nanoparticles can be achieved by either passive or active targeting. Active targeting of a therapeutic agent is achieved by conjugating the therapeutic agent or the carrier system to a tissue or cell-specific ligand (Lamprecht et al., Biodegradable nanoparticles for targeted drug delivery in treatment of inflammatory bowel disease, J Pharmacol Exp Ther. 299:775-81, 2002). Passive targeting is achieved by coupling the therapeutic agent to a macromolecule that passively reaches the target organ (Monsky W L et al., Augmentation of transvascular transport of macromolecules and nanoparticles in tumors using vascular endothelial growth factor, Cancer Res. 59:4129-35, 1999). Drugs encapsulated in nanoparticles or drugs coupled to macromolecules, such as high molecular weight polymers, passively target tumor tissue through the enhanced permeation and retention effect (Maeda H, The enhanced permeability and retention (EPR) effect in tumor vasculature: the key role of tumor-selective macromolecular drug targeting, Adv Enzyme Regul. 41:189-207, 2001; Sahoo S K et al., Pegylated zinc protoporphyrin: a water-soluble heme oxygenase inhibitor with tumor-targeting capacity, Bioconjugate Chem. 13:1031-8, 2002).

In one application, RNA nanoparticles can be used in connection with RNA interference (RNAi) and the delivery of small interfering RNAs (siRNA). RNAi is a cellular process that regulates gene expression post-transcriptionally. Through the foreign introduction of small interfering RNAs (siRNA), this pathway can be mobilized allowing for the regulation of genes that contribute to the diseased state. While RNA interference continues to hold incredible potential, numerous challenges associated with the application of RNAi technology must be addressed before it can be made into a viable therapy. The most prominent challenges include transporting, targeting several genes inside the same diseased cell, and stabilizing short interfering RNAs (siRNAs). In order to simultaneously target several genes inside the same diseased cell with multiple siRNAs, those siRNAs need to be co-delivered in a controlled fashion, e.g., co-delivery components initially having split or separated functionalities which in the intracellular environment interact to produce one or more desired functionalities, such as siRNA gene silencing. RNA nanotechnology can provide novel technologies for delivery siRNAs that involve the introduction of programmable RNA-based nanoparticles that can be functionalized to work with diverse range of therapeutics.

RNA-based nanoparticles have certain advantages over prior technologies. First, RNA is biocompatible and presents a safe vehicle for the delivery of drugs and therapeutics. Second, RNA has an inherent ability to self-assemble to include various functionalities, including, RNA or DNA aptamers, fluorescent dyes, small molecules, and proteins. In addition, RNA/DNA hybrid systems can be used to split RNA-based functionalities which only become activated when the RNA/DNA hybrid system re-associates to form double stranded RNA. Furthermore, RNA-based nanoparticles made of unmodified nucleotides can be synthesized directly via run-off transcription, making their ease of synthesis and cost of production attractive for scaled-up production. Nevertheless, current RNA-based nanoparticles have certain disadvantages as well, including unwanted immunogenicity, lack of stability, low and/or irregular production yields, and difficulty of predicting optimal designs.

Thus, further improvements in the design and implementation of RNA-based nanoparticles are needed in order to achieve more robust biomedical applications, including for example, in RNA interference. The present invention seeks to solve these problems.

### SUMMARY OF THE INVENTION

The present invention relates, in part, to an activatable nanoparticle system comprising at least two interacting cognate nanoparticles having one or more split (and initially inactive) functionalities, wherein said split functionalities are activated as a result of ssRNA-toehold-dependent interaction of the at least two cognate nanoparticles. In certain embodiments, the interacting cognate nanoparticles may each be a RNA/DNA hybrid duplex comprising reverse complement RNA and DNA sequences as between the two nanoparticles, and wherein the RNA strand on each of the nanoparticles further comprises a ssRNA toehold, e.g., 2, 4, 6, 8, 10, 12, or more nucleotides in length. In other embodiments, the cognate nanoparticles may comprise a first nanoparticle having an RNA or DNA “core” nanoscaffold (e.g., a nanoring or nanocube) with functionalized hybrid duplex arms with ssRNA toeholds and a second nanoparticle in the form of a cognate RNA/DNA hybrid duplex with reverse complementary sequences and an ssRNA toehold. The one or more functionalities are initially “split” in an inactive form as between the first and second nanoparticle through their physical separation on the two interacting particles (e.g., separated strands of functional duplex RNA). However, the functionalities become activated upon their reassociation vis-à-vis an ssRNA-toehold-dependent interaction between the nanoparticles. Such split functionalities may include transcriptional activation through the formation of a DNA duplex comprising an (initially split) promoter sequence, the formation of an RNA duplex that functions as a Dicer substrate for siRNA-based gene silencing, the formation of an optical response (e.g., FRET) formed through the joining of initially split optical response moieties (e.g., Alexa 488 and Alexa 546),and reassembly of double-stranded nucleic acid-based aptamers (e.g., Malachite Green isothiocyanate) initially split between the initially inactive interacting cognate nanoparticles.

In certain embodiments, the one or more functionalities are formed as a result of the reassociation of initially separated functionalities that become joined together (e.g., a functional RNA or DNA duplex) and wherein such reassociation is facilitated and/or initiated by way of the reverse complement ssRNA toeholds on each of the interacting nanoparticles.

It was surprisingly and unexpectedly discovered and/or theorized that the herein described activatable nanoparticle system comprising two or more cognate interactable nanoparticles with ssRNA toeholds and split complementary functionalities possessed a number of advantages as compared to corresponding nanoparticles having single strand DNA (“ssDNA”) toehold sequences, including greater stability (both chemical and thermal stability), less immunogenicity, smaller size, and greater production yields, e.g., by run-off transcription, and can more readily and reliably be designed through computational methods, as compared to other RNA/DNA hybrid nanoparticles with single stranded DNA toeholds, such as those described in PCT/US2015/029553 (which is incorporated herein by reference).

Without being bound by theory, it is believed that immunogenicity of RNA nanoparticles is a function of the particles' shape and size, among other properties. And, the R/DNA hybrid nanoparticles comprising ssRNA toehold sequences described herein are smaller and more regular shaped than R/DNA nanoparticles comprising ssDNA toehold sequence. Thus, it is theorized that the more regularly shaped and smaller sized RNA nanoparticles described herein would result in RNA-based nanoparticles with less immunogenicity, among other advantages including higher production yield.

Thus, in one aspect, the present invention provides RNA-based nanoparticles comprising ssRNA toeholds which represent an improvement over previously described RNA-based nanoparticles comprising ssDNA toeholds, e.g., as described in PCT/US2015/029553, which is incorporated herein by reference.

Although RNA nanoparticles comprising ssDNA toeholds were the subject of the inventors' prior application (PCT/US2015/029553), the use of ssRNA toehold sequences was not previously contemplated, described, or used. In part, ssRNA toeholds were not contemplated, described, or used because ssRNA toeholds would not have been a logical modification given that single stranded RNA is well known to form secondary structures. It would have been assumed that such secondary structures would have formed in the single stranded RNA toehold sequences, which would have prevented their functioning in the reassociation process.

Accordingly, in certain aspects, the present invention describes the design and synthesis of various RNA nanoparticles. In preferred embodiments, the nanoparticles of the invention comprise RNA/DNA hybrid systems that utility single stranded RNA toehold sequence to facilitate their assembly and/or reassociation with cognate RNA/DNA hybrid molecules.

The RNA nanoparticles of the present invention can be designed to self-assemble into predefined size and geometric shapes, in particular rings, squares, cubes, or a three dimensional RNA polyhedral cage any of which can carry multiple components including molecules for specific cell recognition, image detection, and therapeutic treatment, and to encapsulate small therapeutic molecules inside their cages and release them upon being triggered by small ligands. In particular, the RNA nanoparticles of the present invention can be further designed to be spatially addressable by optimizing the location of 3-tail connectors in the variable stem and thus controlling the positioning of the biotin within the cage. This allows either the encapsulation of proteins inside the cage or their attachment to the outside forming aggregates of cages. Like proteins and DNA, RNA can potentially lead to stable polyhedral RNA architectures for use as carriers in nano-medicine and synthetic biology.

In other aspects, the RNA nanoparticles of the present invention can be designed to be functionalization with various functional molecules or elements, including multiple short interfering RNAs for combinatorial RNA interference, RNA aptamers, fluorescent dyes, and proteins.

In other aspects, the invention provides RNA/DNA hybrid nanoparticles with conditionally active multiple split functionalities.

In another aspect, the invention features an R/DNA chimeric or hybrid nanoparticle (R/DNA NP) comprising one or more functionalities and including one or more ssRNA toeholds.

Another aspect of the invention provides an R/DNA chimeric nanoparticle (R/DNA NP) having a form of a tube, ring, cube, and the like and having one or more functionalities and including one or more ssRNA toeholds.

In one embodiment, the R/DNA NP possesses one or more RNA-DNA hybrid arm extensions, wherein at least one extension includes an ssRNA toehold. Optionally, one or more of the RNA-DNA hybrid arm extensions is capable of triggered release, formation and/or activation of a dsRNA.

In one embodiment, the functionalities comprise one or more agents. In other embodiments, the agents are selected from one or more of the group consisting of inhibitory nucleic acids, fluorescent dyes, small molecules, RNA-DNA hybrids with split functionalities, split lipase, split GFP, proteins, therapeutic agents and imaging agents. In a related embodiment, the inhibitory nucleic acids are selected from the group consisting of siRNAs, RNA or DNA aptamers and ribozymes.

In one embodiment, the one or more agents are the same. In another embodiment, the one or more agents are different.

In one embodiment, the R/DNA nanoparticle comprises at least two chimeric nanoparticles. In another embodiment, the first chimeric nanoparticle comprises a first DNA oligonucleotide and a complementary first RNA oligonucleotide comprising the one or more functionalities, and the second chimeric nanoparticle comprises a second DNA oligonucleotide and a complementary second RNA oligonucleotide comprising the one or more functionalities. In a further embodiment, the first chimeric nanoparticle comprises the sense strand of double stranded (ds) RNA and the second chimeric nanoparticle comprises the antisense strand of dsRNA. In a further embodiment, the first RNA oligonucleotide and the second RNA oligonucleotide comprise ssRNA toehold sequences which extend past the first and second DNA oligonucelotides and are complementary to one another.

In another aspect, the invention provides RNA-DNA and DNA-RNA hybrid nanostructures, e.g., nanocubes, consisting of either RNA or DNA cores (composed of a plurality of strands, e.g., 2, 3, 4, 5, or 6 or more strands of RNA or DNA oligonucleotides) with attached RNA-DNA hybrid duplex “arms” may be used to conditionally activate different functionalities whereby the functional entity or molecule, e.g., Dicer Substrate RNAs, or DS RNAs, RNA aptamers, FRET pair of dyes) is split into two RNA-DNA hybrids, i.e., where a first hybrid is associated with the RNA or DNA nanocube, and a second cognate hybrid is a free RNA-DNA hybrid molecule, both of which are inactive in the hybrid state. In preferred embodiments, the RNA component of the DNA-RNA hybrids further comprise single strand RNA “toeholds” which are complimentary as between the nanostructure/nanocube hybrids and freely existing cognate DNA/RNA hybrid molecules and which may interact with one another and trigger the reassociation process when both of the cognate hybrids are present in close proximity. The reassociation process results in strand swapping to form DNA-DNA and RNA-RNA hybrids, thereby releasing the split functionalities and restoring and/or triggering their function (e.g., Dicer processing to trigger RNA interference). The RNA “toehold” sequences greatly facilitate the reassociation process.

In certain embodiments, the RNA toehold sequences are less than 4 nucleotides in length. In other embodiments, the RNA toehold sequences are 4 or more nucleotides in length. In still other embodiments, the RNA toehold sequences are between 4 and 6 nucleotides, or between 5 and 7 nucleotides, or between 6 and 8 nucleotides, or between 7 and 9 nucleotides, or between 8 and 10 nucleotides, or between 9 and 11 nucleotides, or between 10 and 12 nucleotides, or between 11 and 13 nucleotides, or between 12 and 14 nucleotides, or between 13 and 15 nucleotides, or between 14 and 16 nucleotides, or more than 16 nucleotides. In other embodiments, the single stranded RNA toehold sequences are 3, 4, 5, 6, 7, 8, 9, 10, 11, 12, 13, 14, 15, 16, 17, 18, 19, or 20 nucleotides in length.

In one aspect, the present invention is directed to an improvement over the RNA/DNA nanostructures described in the inventors' earlier application, PCT/US2015/029553 (WO2015/171827), which is incorporated herein by reference in its entirety. The improvement in part relates to the use of ssRNA toehold sequences in place the earlier-described ssDNA toehold sequences.

Thus, in one aspect, the present invention relates to using RNA oligonucleotides which self-assemble to form a RNA nanoparticle scaffold, which further comprises RNA oligonucleotide “arms” which are further annealed to cognate DNA oligonucleotides, thereby forming a nanocube structure comprised of an RNA “core” scaffold comprising RNA-DNA hybrid arms with ssRNA toeholds. Such RNA nanocubes having RNA-DNA hybrid arms can then be mixed (i.e., allowed to associate) with cognate free DNA-RNA hybrid molecules (where each DNA oligonucleotide strand of the free DNA-RNA hybrid molecule is antisense to the sequence of the DNA sequence of the RNA-DNA sequences of the hybrid arms of the nanocube, and vice versa), with such mixing occurring, for example, in solution, in cell culture, following delivery, etc. Such mixing of RNA nanocubes possessing DNA-RNA hybrid arms with free RNA-DNA molecules can promote dissociation and subsequent annealing of arm structures, resulting in RNA nanocubes having RNA-RNA (dsRNA) arms and free dsDNA molecules, thereby activating the innate functionalities of the ds molecules.

For example, in certain embodiments, the reassembled dsRNA arms of the RNA nanocubes can be cleaved by Dicer and serve as active RNAi agents (e.g., siRNAs, including, e.g., DsiRNAs), or otherwise serve to activate the RNA interference pathway.

In preferred embodiments, the RNA component of the DNA-RNA hybrids further comprise single strand RNA “toeholds” which are complimentary as between the nanocube hybrids and the freely existing cognate hybrids and which may interact and trigger the reassociation process when both of the cognate hybrids are present in close proximity.

Advantages associated with the inclusion of hybrid arms in an RNA nanocube structure, as compared to entirely RNA nanocubes possessing dsRNA arms, include: reduced immunogenicity, enhanced stability and the functionality of the structures provides the ability to form an initially inactive particle (the RNA scaffold nanocube with hybrid arms) that is then activated for RNAi activity only upon association with a DNA-RNA hybrid molecule that presents strands capable of annealing to corresponding DNA and RNA oligonucleotides of the hybrid arms (where each DNA oligonucleotide strand of the free DNA-RNA hybrid molecule is antisense to the sequence of the DNA sequence of the RNA-DNA sequences of the hybrid arms of the nanocube, and vice versa, therefore driving respective formation of dsDNA and dsRNA duplexes).

Further advantages associated with DNA nanocube scaffolds possessing hybrid arms include: reduced immunogenicity, enhanced stability, a scaffold that can be even more readily labeled (e.g., fluorescently labeled) than an RNA scaffold structure, the functionality of the structures provides the ability to form an initially inactive particle (the DNA scaffold nanocube with hybrid arms) that then releases activated RNAi agents only upon association with a DNA-RNA hybrid molecule that presents strands capable of annealing to corresponding DNA and RNA oligonucleotides of the hybrid arms (where each DNA oligonucleotide strand of the free DNA-RNA hybrid molecule is antisense to the sequence of the DNA sequence of the RNA-DNA sequences of the hybrid arms of the nanocube, and vice versa, therefore driving respective formation of dsDNA and dsRNA duplexes), the fact that such structures release a free dsRNA (e.g., an RNAi agent, e.g., siRNA or DsiRNA).

Accordingly, in certain aspects, the present invention relates to an activatable nanoparticle system comprising at least two cognate interactable nanoparticles each comprising at least one duplex of DNA and/or RNA each with an ssRNA toehold region, wherein the at least two cognate interactable nanoparticles comprise reverse complement sequences such that when the particles interact, the strands of a first nanoparticle separate and reassociate with the cognate reverse complement strand of the second nanoparticle and wherein the strand reassociation process is facilitated by the presence of the cognate ssRNA toeholds having reverse complement sequences on each of the nanoparticles of the system.

In some embodiments, the activatable nanoparticles comprise DNA nanocubes. The DNA nanocubes may include at least one single-stranded DNA arm, or at least two single-stranded DNA arms, or at least three single-stranded DNA arms, or at least four single-stranded DNA arms, or at least five single-stranded DNA arms, each of which have the capacity to anneal to a cognate or complimentary RNA oligonucleotide. In preferred embodiments, the RNA component of the DNA-RNA hybrids further comprise single strand RNA “toeholds” which are complimentary as between the nanocube hybrids and the freely existing cognate hybrids and which may interact and trigger the reassociation process when both of the cognate hybrids are present in close proximity.

In other embodiments, the DNA nanocubes may include at least one single-stranded RNA arm, or at least two single-stranded RNA arms, or at least three single-stranded RNA arms, or at least four single-stranded RNA arms, or at least five single-stranded RNA arms, each of which have the capacity to anneal to a cognate or complimentary DNA oligonucleotide. In preferred embodiments, the RNA component of the DNA-RNA hybrids further comprise single strand RNA “toeholds” which are complimentary as between the nanocube hybrids and the freely existing cognate hybrids and which may interact and trigger the reassociation process when both of the cognate hybrids are present in close proximity.

In other embodiments, the activatable nanoparticles comprise RNA nanocubes. The RNA nanocubes may include at least one single-stranded RNA arm, or at least two single-stranded RNA arms, or at least three single-stranded RNA arms, or at least four single-stranded RNA arms, or at least five single-stranded RNA arms, each of which have the capacity to anneal to a cognate or complimentary DNA oligonucleotide. In preferred embodiments, the RNA component of the DNA-RNA hybrids further comprise single strand RNA “toeholds” which are complimentary as between the nanocube hybrids and the freely existing cognate hybrids and which may interact and trigger the reassociation process when both of the cognate hybrids are present in close proximity.

In other embodiments, the RNA nanocubes may include at least one single-stranded DNA arm, or at least two single-stranded DNA arms, or at least three single-stranded DNA arms, or at least four single-stranded DNA arms, or at least five single-stranded DNA arms, each of which have the capacity to anneal to a cognate or complimentary RNA oligonucleotide. In preferred embodiments, the RNA component of the DNA-RNA hybrids further comprise single strand RNA “toeholds” which are complimentary as between the nanocube hybrids and the freely existing cognate hybrids and which may interact and trigger the reassociation process when both of the cognate hybrids are present in close proximity.

In one embodiment, the functionalities include one or more sense or antisense strands of at least one RNAi agent.

In another embodiment, the nanocube includes six single-stranded DNA or RNA arms. Optionally, the nanocube includes one, two, three, four, five, six, or more oligonucleotide strands or functional or duplex arms. In preferred embodiments, the nanoparticles comprise six functional duplex arms. In preferred embodiments, each of the duplex arms comprises a ssRNA toehold for interaction with a cognate nanoparticle.

In another embodiment, the RNA-based nanostructures of the invention deliver siRNA which inhibits a target RNA. In a further embodiment, the target RNA is one which produces a therapeutically beneficial result when inhibited. In another further embodiment, the target RNA comprises an RNA that encodes a protein involved in a disease process or a portion thereof. In a further related embodiment of any one of the above aspects, the target RNA encodes an apoptosis inhibitor protein. In another further related embodiment of any one of the above aspects, the target RNA is a pathogenic RNA genome, an RNA transcript derived from the genome of the pathogenic agent, or a portion thereof. In one embodiment, the pathogenic agent is a virus, a bacteria, a fungus, or a parasite. In another embodiment, the target RNA is a viral RNA genome or a portion thereof.

The invention also features a composition comprising an R/DNA NP of any one of the above aspects.

The invention also features a pharmaceutical composition comprising an R/DNA NP of any one of the above aspects.

In one embodiment, the pharmaceutical composition further comprises a pharmaceutically acceptable excipient, carrier, or diluent.

In another embodiment, the pharmaceutical composition is formulated for the treatment of a disease.

In still another embodiment, the pharmaceutical composition is formulated for the treatment of an infection by a pathogenic agent. In another related embodiment, the pathogenic agent is a virus, a bacterium, a fungus, or a parasite.

The invention also features a method of inhibiting or reducing the expression of a target gene in a cell comprising contacting the cell with a therapeutically effective amount of the R/DNA NP of any of the above aspects or embodiments, or the composition of any one of the above aspects or embodiments.

The invention also features a method of killing a pathogen infected cell comprising contacting the cell with a therapeutically effective amount of the R/DNA NP of any one of the above aspects or embodiments or the composition of any one of the above aspects or embodiments.

The invention also features a method of inhibiting replication of a pathogen in a cell comprising contacting the cell with a therapeutically effective amount of the R/DNA NP of any one of the above aspects or embodiments or the composition of any one of the above aspects or embodiments.

In one embodiment, the cell is in a subject.

The invention also features a method of reducing pathogenic burden in a subject comprising administering a therapeutically effective amount of the R/DNA NP of any one of the above aspects or embodiments or the composition of any one of the above aspects or embodiments.

In one embodiment, the subject is at risk of developing a pathogenic infection and/or a tumor.

In another embodiment, the subject is diagnosed with having a pathogenic infection and/or a tumor.

The invention also features a method of treating or preventing a pathogenic infection and/or a tumor in a subject comprising administering a therapeutically effective amount of the R/DNA NP of any one of the above aspects or embodiments or the composition of any one of the above aspects or embodiments.

In one embodiment, the method reduces the pathogenic burden, thereby treating or preventing the pathogenic infection. In another embodiment, the method induces death in infected cell, thereby treating or preventing the pathogenic infection.

In one embodiment, the subject is a mammal. In another embodiment, the subject is a human.

In one embodiment, the pathogen is a virus, bacteria, fungus, or parasite.

In another embodiment of any one of the above aspects or embodiments, the method further comprises contacting the cell with a therapeutically effective amount of a second therapeutic agent or administering a therapeutically effective amount of the second therapeutic agent to the subject.

In one embodiment, the second therapeutic agent treats the pathogenic infection or the symptoms associated with the pathogenic infection.

The invention also features a method of killing a neoplastic cell comprising contacting the cancer cell with a therapeutically effective amount of the of the R/DNA NP of any one of the above aspects or embodiments or the composition of any one of the above aspects or embodiments, thereby killing the neoplastic cell.

The invention also features a method of treating a subject having a neoplasia, the method comprising administering to a subject a therapeutically effective amount of the R/DNA NP of any one of the above aspects or embodiments or the composition of any one of the above aspects or embodiments, thereby treating the subject.

In one embodiment, the neoplastic cell is a cancer cell which is present in a solid tumor.

In another embodiment, the method further comprises contacting the cell with a therapeutically effective amount of a second therapeutic agent or administering a therapeutically effective amount of the second therapeutic agent to the subject.

In one embodiment, the second therapeutic agent is an anti-cancer agent.

The invention also features a kit comprising the R/DNA NP of any one of the above aspects or embodiments or the composition of any one of the above aspects or embodiments.

In one aspect, the kit further comprises a second therapeutic agent.

The invention also features an algorithm for the computational prediction of RNA/DNA hybrid re-association and RNA secondary structures.

In certain aspects, the invention relates to an activatable nanoparticle system comprising one or more split functionalities comprising a first inactive nanoparticle comprising a first set of DNA and/or RNA strands and a first ssRNA toehold and a second inactive nanoparticle comprising a second set of DNA and/or RNA strands and a second ssRNA toehold, wherein the strands of the first inactive nanoparticle are the reverse complements of the strands of the second inactive nanoparticle, wherein the first and second inactive nanoparticles are capable of undergoing reassociation of their strands to produce one or more functionalities, and wherein the reassociation of strands is triggered by the interaction of the first and second ssRNA toeholds.

In other aspects, the invention relates to a method of triggering one or more functionalities in a cell comprising:

(a) administering an effective amount of a first inactive nanoparticle comprising a first set of DNA and/or RNA strands and a first ssRNA toehold;

(b) administering an effective amount of a second inactive nanoparticle comprising a second set of DNA and/or RNA strands and a second ssRNA toehold;

wherein the strands of the first inactive nanoparticle are the reverse complements of the strands of the second inactive nanoparticle,

wherein the first and second inactive nanoparticles are capable of undergoing reassociation of their strands to produce one or more functionalities, and

wherein the reassociation of strands of the first and second nanoparticles is triggered by the interaction of the first and second ssRNA toeholds.

In still other aspects, the invention relates to a method of inhibiting a target gene in a cell comprising:

(a) administering an effective amount of a first inactive nanoparticle comprising a first set of DNA and/or RNA strands and a first ssRNA toehold;

(b) administering an effective amount of a second inactive nanoparticle comprising a second set of DNA and/or RNA strands and a second ssRNA toehold;

wherein the strands of the first inactive nanoparticle are the reverse complements of the strands of the second inactive nanoparticle,

wherein the first and second inactive nanoparticles are capable of undergoing reassociation of their strands to produce one or more functionalities which inhibit a target gene in the cell, and

wherein the reassociation of strands of the first and second nanoparticles is triggered by the interaction of the first and second ssRNA toeholds.

In various embodiments, the one or more split functionalities is selected from the group consisting split transcription, split aptamer, split optical response, and split Dicer substrate.

In other embodiments, the ssRNA toeholds are 2, 4, 6, 8, 10, or 12 nucleotides. In still other embodiments, the ssRNA toeholds are at least 4 nucleotides.

In other embodiments, the ssRNA toeholds impart greater stability (both chemical and thermal stability), less immunogenicity, smaller size, and greater production yields by run-off transcription as compared to nanoparticles with ssDNA toeholds.

In other embodiment still, the first or second nanoparticle is a nanoring, nanotube, or nanocube comprising one or more hybrid duplex arms comprising the first or second ssRNA toehold.

In various embodiments, the first or second nanoparticle is an RNA/DNA duplex comprising the first or second ssRNA toehold.

In still other embodiments, the split Dicer substrate inhibits a target gene.

Other aspects of the invention are described in, or are obvious from, the following disclosure, and are within the ambit of the invention.

### BRIEF DESCRIPTION OF THE DRAWINGS

FIG. 1A through 1G show initiation of the re-association of two interacting split-function cognate nanoparticles (each are cognate RNA/DNA hybrids) by RNA toeholds that leads to activation of the resulting functions of RNAi and FRET (optical response signal as a marker of completed strand reassociation).

FIG. 1A is a schematic representation of re-association for cognate interacting nanoparticles (e.g., RNA/DNA hybrids) with 10 nts RNA toeholds. However, in various embodiments, the length of the RNA toeholds may vary, including 2, 4, 6, 8, 10, or 12 or more nucleotides.

FIG. 1B provides a native-PAGE that demonstrates the re-association (1 hour at 37° C.) of fluorescently labeled hybrids with 2, 4, 6, and 8 nts RNA toeholds (at 1 μM final).

FIG. 1C: In vitro analysis of hybrids with 2, 4, 6, and 8 nts RNA toeholds re-association (but no Dicing) using FRET. Emission of Alexa-488 was measured every 30 sec. Hybrids containing Alexa-546 were added 2 mins after the incubation.

FIG. 1D: FRET experiments: cells were co-transfected with cognate hybrids, with 8 nts RNA toeholds (100 nM final), labeled with Alexa546 and Alexa488 and images were taken on the next day. Image numbers correspond to: (1)—differential interference contrast images, (2)—Alexa488 emission, (3)—Alexa546 emission, (4)—bleed-through corrected FRET image, (5) 3D chart representation of zoomed fragment indicated by a white box of bleed-through corrected FRET image.

FIG. 1E: GFP knockdown assays for GFP expressing human breast cancer cells. Three days after the co-transfection of cells with RNA/DNA hybrids with 2, 4, 6, and 8 nts RNA toeholds, GFP expression was statistically analyzed with flow cytometry experiments. Hybrid concentrations (in nM) used in the silencing experiments are indicated.

FIG. 1F: FRET analysis of concentration dependent re-association of hybrids with 2 nts ssRNA toeholds. The hybrids were incubated at the indicated concentrations for 5 hours in a water bath at 37° C. Upon incubation, they were diluted to the same final concentration of 90 nM final and then subjected to fluorescence analysis. Error bars denote+/−S.E.M. RNA toeholds initiate the re-association of RNA/DNA hybrids that leads to activation of RNAi Schematic representation of re-association for RNA/DNA hybrids with 8-nts RNA toeholds. Depicted is total staining (with Ethidium Bromide) native-PAGE experiments that demonstrate the re-association of labeled hybrids with 2-, 4-, 6-, and 8-nts RNA toeholds (at 1 μM final). The hybrids with 4-nts ssRNA toeholds re-associate partially and hybrids with 2-nts do not re-associate at all.

FIG. 1G: Provides a generalized schematic depicting an activatable nanoparticle system described herein. The top of the schematic depicts a first nanoparticle (“NP1”) corresponding to a first DNA/RNA hybrid having an ssRNA toehold sequence (e.g., 2, 4, 6, 8, 10, or 12 nucleotides). As shown, the first nanoparticle is combined with a second nanoparticle (“NP”), e.g., inside a target cell, wherein the second nanoparticle is also a hybrid comprising the cognate reverse complement DNA and RNA sequences, and include the reverse complement single stand RNA toehold. During the interaction between the nanoparticles, the reverse complement ssRNA strands hybridize, and then through a process of branch migration, the RNA strands NP1 and NP2 form an RNA duplex. Concomitantly, the DNA strands of NP1 and NP2 form a DNA duplex. The RNA duplex provides for a first functionality, i.e., a Dicer substrate and/or siRNA molecule for gene silencing of a target sequence. The DNA duplex forms an optical marker resulting from the interaction of the split optical response elements (e.g., FRET fluorophores) which are brought together only in the DNA duplex. The optical response can be used to monitor the interaction of NP1 and NP2 and the successful reassociation of their strands. The figure also lists other “exemplary embodiments” for the interacting NPT and NP2, which can include nanorings and nanocubes comprising split-function hybrids with ssRNA toeholds as NP1, along with cognate split-function hybrid duplexes with ssRNA toeholds as NP2. Split functionalities can include, for example, (a) split siRNA or Dicer substrates, (b) split promoter elements for transcription, (c) split optical response elements (e.g., FRET), (d) split aptamer elements (e.g., Malachite green), among others. The figure also depicts in the lower drawing an activatable nanoparticle system than includes functionalized nanorings decorated with six DNA/RNA hybrids with a ssRNA toehold region, and cognate DNA/RNA hybrids with ssRNA toeholds having the reverse complement sequences. Once combined and allowed to interact, the RNA strand from the cognate hybrids forms a duplex with the RNA strand of the nanoring. Dicing the decorated nanoring produces siRNA molecules.

FIG. 2A through 2C show inhibition of HIV-1 gene expression by an activatable nanoparticle system comprising a pair of cognate RNA/DNA hybrids each with 8 nts RNA toeholds. Hela cells were transfected with an HIV-1 infectious clone, pGluc and nucleic acids as indicated. The control dsRNA targets mRNA of the cellular glutathione S-transferase P1 gene.

FIG. 2A: Transfected Hela cell samples were harvested after 48 h. Cell lysates were probed by western blotting for HIV-1 Gag protein and the constitutively-expressed cellular protein GAPDH.

FIG. 2B: Band intensities of the western blotting of FIG. 2A were measured and Gag expression was calculated as the sum of p55Gag, p41 and CA divided by GAPDH for each sample. These values were expressed relative to HIV-1 in the absence of nucleic acid and the results of four independent experiments were plotted+/−S.E.M.

FIG. 2C: Samples of culture media were assayed for the RT activity as an indicator of HIV-1 particle release and for the presence of Gaussia luciferase. The RT value was normalized to the Gaussia luciferase value and expressed relative to HIV-1 in the absence of nucleic acids and the results of four independent experiments were plotted. Hybrid concentrations (in nM) used in these gene silencing experiments are indicated. Error bars denote+/−S.E.M FIG. 3A shows RNA toeholds initiate the re-association of interacting nanoparticles of an activatable nanoparticle system comprising (a) a nanoring decorated with six RNA/DNA hybrid arms with an ssRNA toehold (e.g., 10 nts), and (b) six cognate RNA/DNA hybrids comprising the reverse complement sequences including the cognate ssRNA toehold (10 nts).

FIG. 3B: Co-transcriptional assembly of functionalized nanorings (NR) was allowed to occur (at 37° C. for 4 hours). Gel purified NR with six hybrids visualized with total staining native-PAGE (left) and re-association (1 hour at 37° C.) of non-labeled NR with six hybrids (with 8, 6, 4, 2 nts RNA toeholds) traced through the incorporation of fluorescently labeled (with Alexa488 RNA) strands of cognate to NR hybrids. DNA strands are not labeled. The trace amounts of fluorescence at the duplex site of the 8 nt NR lane can be attributed to the excess of hybrid duplexes added to NR.

FIG. 3C: Intracellular re-association of hybrid nanorings (10 nM) and cognate hybrids triggers the GFP silencing. Three days after the co-transfection of cells with nanorings decorated with hybrids and cognate hybrids, GFP silencing was confirmed by fluorescent microscopy.

FIG. 3D: GFP silencing of FIG. 3C was confirmed by statistically analyzed with flow cytometry experiments. Error bars denote+/−S.E.M.

FIG. 4A through 4D shows in silico prediction of RNA/DNA hybrids re-association initiated by ssRNA toeholds.

FIG. 4A: Predicted free energies for individual RNA/DNA hybrids with different lengths of ssRNA toeholds (2, 4, 6, and 8 nts), for their transition states and for their re-associated states (dsRNAs and dsDNAs). Shown are results for concentrations of 0.03, 0.25, 1, 5 and 10 μmol/l.

FIG. 4B: Predicted free energies of toehold activation for each transition state model. For high energies of activation the non-reassociated state becomes kinetically trapped. This is the case for 2 and 4 nt toeholds, unless high concentrations are employed. The model of a transition state consists of the non-reassociated RNA/DNA hybrid helices combined with binding of the RNA toeholds. The shown labels indicate toehold lengths.

FIG. 4C: The circular plot depicts intra-strand base pairs as blue arcs and inter-strand base pairs as red arcs. Nanorings are strands A-F and cognate DNAs are strands G-L. Predicted ring scaffold for RNA sequences without toehold arms.

FIG. 4D: The circular plot depicts intra-strand base pairs as blue arcs and inter-strand base pairs as red arcs. Nanorings are strands A-F and cognate DNAs are strands G-L. Predicted ring with hybrid arms and bound cognate DNAs (before re-association).

FIG. 5 shows relative yields of co-transcriptionally assembled RNA nanorings functionalized with RNA-DNA hybrids measured after native-PAGE purification and recovery. Each nanoring was functionalized with six RNA-DNA hybrids of varying ssRNA toehold lengths (8-nts, 6-nts, 4-nts, and 2-nts in length) and is compared with ssDNA toehold.

FIG. 6 shows in silico predictions of re-association of RNA/DNA hybrids initiated by 2-, 4-, 6-, and 8-nts ssRNA toeholds. RNA strands are depicted as red segments; DNA strands are shown in blue. Each base pair corresponds to an arc shown in black.

### DETAILED DESCRIPTION OF THE INVENTION

The present invention relates, in part, to an activatable nanoparticle system comprising at least two interacting cognate nanoparticles having one or more split (and initially inactive) functionalities, wherein said split functionalities are activated as a result of ssRNA-toehold-dependent interaction of the at least two cognate nanoparticles. In certain embodiments, the interacting cognate nanoparticles may each be a RNA/DNA hybrid duplex comprising reverse complement RNA and DNA sequences as between the two nanoparticles, and wherein the RNA strand on each of the nanoparticles further comprises an ssRNA toehold, e.g., 2, 4, 6, 8, 10, 12, or more nucleotides in length. In other embodiments, the cognate nanoparticles may comprise a first nanoparticle having an RNA or DNA “core” nanoscaffold (e.g., a nanoring or nanocube) with functionalized hybrid duplex arms with ssRNA toeholds and a second nanoparticle in the form of a cognate RNA/DNA hybrid duplex with reverse complementary sequences and an ssRNA toehold. The one or more functionalities are initially “split” in an inactive form as between the first and second nanoparticle through their physical separation on the two interacting particles (e.g., separated strands of functional duplex RNA). However, the functionalities become activated upon their reassociation vis-à-vis an ssRNA-toehold-dependent interaction between the nanoparticles. Such split functionalities may include transcriptional activation through the formation of a DNA duplex comprising an (initially split) promoter sequence, the formation of an RNA duplex that functions as a Dicer substrate for siRNA-based gene silencing, the formation of an optical response (e.g., FRET) formed through the joining of initially split optical response moieties (e.g., Alexa 488 and Alexa 546),and reassembly of double-stranded nucleic acid-based aptamers (e.g., Malachite Green isothiocyanate) initially split between the initially inactive interacting cognate nanoparticles.

Without being bound by theory, it is believed that immunogenicity of RNA nanoparticles is a function of the particles' shape and size, among other properties. And, the R/DNA hybrid nanoparticles comprising ssRNA toehold sequences described herein are smaller and more regular shaped than R/DNA nanoparticles comprising ssDNA toehold sequence. Thus, it is theorized that the more regularly shaped and smaller sized RNA nanoparticles described herein would result in RNA-based nanoparticles with less immunogenicity, among other advantages including higher production yield.

Thus, in one aspect, the present invention provides RNA-based nanoparticles comprising ssRNA toeholds which represent an improvement over previously described RNA-based nanoparticles comprising ssDNA toeholds, e.g., as described in PCT/US2015/029553, which is incorporated herein by reference.

Although RNA nanoparticles comprising ssDNA toeholds were the subject of the inventors' prior application (PCT/US2015/029553), the use of ssRNA toehold sequences was not previously contemplated, described, or used. In part, ssRNA toeholds were not contemplated, described, or used because ssRNA toeholds would not have been a logical modification given that single stranded RNA is well known to form secondary structures. It would have been assumed that such secondary structures would have formed in the single stranded RNA toehold sequences, which would have prevented their functioning in the reassociation process.

The present invention also relates to the continued development of RNA-based nanoparticles, e.g., siRNA nanoscaffolds, using nanorings, nanocubes, and other three dimensional RNA structures, or RNA/DNA hybrid structures. The RNA-based nanoparticles described herein comprise ssRNA toeholds to promote the exchange of hybrid strands of RNA and DNA as between two different RNA/DNA hybrid nanoparticles (e.g., a hybrid RNA/DNA nanoparticle and a cognate or complimentary RNA/DNA hybrid fragment). In certain embodiments, upon re-association between the hybrid molecules vis-à-vis the action of the RNA toeholds, double stranded RNA/RNA molecules are formed, thereby activating various functionalities which are silent when the RNA is complexed with the DNA prior to reassociation. These functionalized polyvalent RNA/DNA nanoparticles are suitable for therapeutic or diagnostic use in a number of diseases or disorders.

The RNA/DNA nanoparticles described herein have the ability to self-assemble into higher order structures, e.g., a nanoring or nanocube and re-associate to release dsRNA. The nanostructures can also be generated as polyvalent, multifunctional nanoparticles that can bind with different agents to effectively kill a target.

Advantageously, the nanoparticles of the instant invention provide a number of improvements over nanoparticles currently available. For example, the RNA/DNA nanoparticles of the invention do not induce a significant immune response as compared to RNA/DNA nanoparticles having DNA toeholds or to protein nanoparticles currently used.

Moreover, the nanoparticles of the invention are smaller than many currently available nanoparticles and therefore allow for increased efficiency of administration. Also, the nanoparticles of the invention with ssRNA toeholds result in higher yield as compared to the nanoparticles comprising ssDNA toeholds. The nanoparticles described herein comprise multiple RNA subunits each of which has the ability to bind an agent. Moreover, multiple different agents can be present within a single nanoparticle. The multivalency of the RNA-based nanoparticles allows for combining therapeutic (e.g. siRNA/miRNA/drug), targeting (e.g. aptamer and chemical ligand) and detection (e.g. radionucleolide, fluorophore) modules, all in one nanoparticle. The presently disclosed RNA nanostructures provide an effective drug delivery vehicle.

In certain embodiments, the present invention exemplifies how the nanoparticle design can achieve cell-targeting and multifunctional properties by splitting the function of a Dicer Substrate RNA (DS RNA), designed to downregulate the production of green fluorescent protein (GFP) (Rose et al Nucleic acids research 2005, 33, 4140-4156) that is stably expressed in model human breast cancer cells (MDA-MB231/GFP). The use of DS RNAs (as opposed to siRNAs) is required to ensure that once inside the cells, the individual hybrids will not be active in the RNAi pathway (Afonin et al Nucleic acids research 2014, 42, 2085-2097). GFP DS RNAs were split between two RNA-DNA hybrids with the DNA strands being shorter than their corresponding complementary RNAs, thus, providing the ssRNA toeholds for further re-association to form DS RNAs to perform gene silencing. This allows for an additional degree of control over when the therapeutic becomes active. Use of ssRNA toeholds overcomes the need to design specific toeholds as it was needed for ssDNA to prevent undesired interactions and formation of internal secondary structures. ssRNA toeholds addressed several of the challenges remaining in using this technology for a clinical application including but not limited to nanoparticle size, undesired interactions, immunogenicity and multifunctionality.

### Definitions

The current invention provides polyvalent R/DNA nanoparticles by using RNA-DNA hybrids and ssRNA toeholds. The polyvalent R/DNA nanoparticles described herein can further comprise therapeutic, diagnostic and/or delivery agents. Further, the polyvalent R/DNA nanoparticles described herein can be used as drug delivery compositions to treat various diseases or conditions.

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a” “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “administering” is meant to refer to a means of providing the composition to the subject in a manner that results in the composition being inside the subject's body. Such an administration can be by any route including, without limitation, subcutaneous, intradermal, intravenous, intra-arterial, intraperitoneal, and intramuscular.

As used herein, the term “functionalities” refers to substances which are capable of being contained in, or attached, to the nanoparticle. In exemplary embodiments, functionality is an agent. Exemplary agents include, for example, prodrugs, diagnostic agents, imaging agents, therapeutic agents, chemotherapeutic agents, pharmaceutical agents, drugs, synthetic organic molecules, proteins, peptides, vitamins, and steroids, siRNAs, RNA or DNA aptamers, fluorescent dyes, small molecules, RNA-DNA hybrids with split functionalities, split lipase, split GFP, split Dicer Substrate (DS), FRET (fluorescence resonance energy transfer) pairs, and proteins.

As used herein, the term “split functionalities” refers to combinable subunits of a functional unit which do not have function in isolation, but which gain function when combined. For instance, the activatable nanoparticle systems of the invention may comprise at least two interactable cognate nanoparticles with one or more split functionalities. The interactable nanoparticles of the system have no function on their own in an initial state, but when combined, allowed to interact (via the ssRNA toehold interaction), and the individual cognate and complementary strands of the interacting nanoparticles allowed to reassociate, the resulting newly formed or reassociated nanoparticles become functionalized. Such split functionalities may include transcriptional activation through the formation of a DNA duplex comprising an (initially split) promoter sequence, the formation of an RNA duplex that functions as a Dicer substrate for siRNA-based gene silencing, the formation of an optical response (e.g., FRET) formed through the joining of initially split optical response moieties (e.g., Alexa 488 and Alexa 546), and reassembly of double-stranded nucleic acid-based aptamers (e.g., Malachite Green isothiocyanate) initially split between the initially inactive interacting cognate nanoparticles.

As used herein, an “aptamer” is an oligonucleotide that is able to specifically bind an analyte of interest other than by base pair hybridization. Aptamers typically comprise DNA or RNA or a mixture of DNA and RNA. Aptamers may be naturally occurring or made by synthetic or recombinant means. The aptamers are typically single stranded, but may also be double stranded or triple stranded. They may comprise naturally occurring nucleotides, nucleotides that have been modified in some way, such as by chemical modification, and unnatural bases, for example 2-aminopurine. See, for example, U.S. Pat. No. 5,840,867. The aptamers may be chemically modified, for example, by the addition of a label, such as a fluorophore, or a by the addition of a molecule that allows the aptamer to be crosslinked to a molecule to which it is bound. Aptamers are of the same “type” if they have the same sequence or are capable of specific binding to the same molecule. The length of the aptamer will vary, but is typically less than about 100 nucleotides. An example of an aptamer may include Malachite green.

As used herein, the term “therapeutic agent” is meant to refer to an agent that is capable of exerting an effect on a target, in vitro or in vivo.

As used herein, the term “chemotherapeutic agent” is meant to include a compound or molecule that can be used to treat or prevent a cancer. A “chemotherapeutic agent” is meant to include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlomaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

As used herein, the term “effective amount” refers to that amount of a therapeutic agent alone that produces the desired effect (such as treatment of a medical condition such as a disease or the like, or alleviation of a symptom such as pain) in a patient. In some aspects, the phrase refers to an amount of therapeutic agent that, when incorporated into a composition of the invention, provides a preventative effect sufficient to prevent or protect an individual from future medical risk associated with a particular disease or disorder. A physician or veterinarian of ordinary skill can readily determine and prescribe the effective amount of the bioactive agent required to treat and/or prevent the progress of the condition.

As used herein, the term “cancer” is used to mean a condition in which a cell in a subject's body undergoes abnormal, uncontrolled proliferation. Thus, “cancer” is a cell-proliferative disorder. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The terms “cancer,” “neoplasm,” and “tumor,” are used interchangeably and in either the singular or plural form, refer to cells that have undergone a malignant transformation that makes them pathological to the host organism. By “neoplastic cell” is meant a cell that is a component of a neoplasia.

As used herein, a “composition” refers to the combination of an active agent (e.g., a polyvalent RNA nanoparticle). The composition additionally can comprise a pharmaceutically acceptable carrier or excipient and/or one or more therapeutic agents for use in vitro or in vivo. A composition may also refer to an activatable nanoparticle system described herein, which may comprise at least two different initially inactive nanoparticles, which when combined are allowed to interact via complementary ssRNA toeholds on each particle, and subsequently are allowed to reassociate into new functional nanoparticles, e.g., a functional duplex RNA Dicer substrate, or a functional DNA duplex bearing partnered optical response elements (e.g., FRET).

As used herein, the term “conjugated” is understood as attached, linked, or otherwise present on a nanoparticle.

As used herein, “disease” is meant to refer to any condition or disorder that damages or interferes with the normal function of a cell, tissue, or organ.

As used herein, “effective amount” is meant to refer to the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

As used herein, “inhibits neoplasia” is meant decreases the propensity of a cell to develop into neoplasia or slows, decreases, or stabilizes the growth or proliferation of a neoplasia.

As used herein, “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

As used herein, “kits” are understood to contain at least the non-standard laboratory reagents of the invention and one or more non-standard laboratory reagents for use in the methods of the invention.

As used herein, the term “nanoparticle” is meant to refer to a particle between 10 nm and 200 nm in size. A nanoparticle according to the invention comprises a ribonucleic acid (RNA). The RNA can be obtained from any source, for example bacteriophages phi 29, HIV, Drosophila, the ribosome, or be a synthetic RNA.

The term “obtaining” is understood herein as manufacturing, purchasing, or otherwise coming into possession of.

The term “oligonucleotide” as used herein includes linear oligomers of nucleotides or analogs thereof, including deoxyribonucleosides, ribonucleosides, and the like. Typically, oligonucleotides range in size from a few monomeric units, e.g., 3-4, to several hundreds of monomeric units. Oligonucleotides can have inhibitory activity or stimulatory activity.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

The term “subject” is intended to include organisms needing treatment. Examples of subjects include mammals, e.g., humans, dogs, cows, horses, pigs, sheep, goats, cats, mice, rabbits, rats, and transgenic non-human animals. In certain embodiments, the subject is a human.

The term “toehold” refers to nucleation site of a domain comprising a nucleic acid sequence designed to initiate hybridization of the domain with a complementary nucleic acid sequence.

The term “ssRNA toehold” refers to nucleation site of a domain comprising a ribonucleic acid sequence designed to initiate hybridization of the domain with a complementary nucleic acid sequence. In certain embodiments, the activatable nanoparticle systems described herein comprises at least two interactable and cognate nanoparticles with reverse complement sequences which interact and reassociate (i.e., swapping of complementary strands or reverse complement strands), wherein said interaction and strand swapping begins with an initiating interaction between the ssRNA toeholds on each of the interacting nanoparticles. The ssRNA toeholds can be of any suitable length, including 1, 2, 3, 4, 5, 6, 7, 8, 9, 10, 11, or 12 nucleotides, or 2, 4, 6, 8, 10, 12 nucleotides, or 2, 4, 6, or 8 nucleotides, or in some embodiments, 2, 4, or 6 nucleotides. In certain embodiments, the ssRNA toeholds have a minimal length of 2 nucleotides. In other embodiments, the ssRNA toeholds have a minimal length of 3 nucleotides. In still embodiments, the ssRNA toeholds have a minimal length of 4 nucleotides. In yet other embodiments, the ssRNA toeholds have a minimal length of 6 nucleotides. In some embodiments, the ssRNA toeholds have a minimal length of 8 nucleotides. In still other embodiments, the ssRNA toeholds have a minimal length of 10 nucleotides. In other embodiments, the ssRNA toeholds have a minimal length of 12 nucleotides.

As used herein, the term “therapeutic agent” includes a drug and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a growth factor, e.g., NGF or GNDF, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-I-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-i-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the term “treated,” “treating” or “treatment” includes the diminishment or alleviation of at least one symptom associated or caused by the state, disorder or disease being treated. A subject that has been treated can exhibit a partial or total alleviation of symptoms (for example, tumor load), or symptoms can remain static following treatment according to the invention. The term “treatment” is intended to encompass prophylaxis, therapy and cure.

As used here, the phrase “5′ or 3′ sticky ends” is meant to refer to the 3′ and/or 5′ protruding ends of DNA or RNA that will bond with complementary sequences of bases. In certain embodiments, the RNA motifs have 5′ or 3′ sticky ends. In certain embodiments, the 5′ or 3′ sticky ends are located in the middle of a helix. According to the invention, the 5′ and 3′ sticky ends can be engineered to be used for self-assembly of the nanorings into an RNA nanotube.

As used here, the phrase “H_ant” is meant to refer to the hybrid containing antisense strand of DS RNA.

As used here, the phrase “Hsen” is meant to refer to the hybrid containing sense strand of DS RNA.

Other definitions appear in context throughout the disclosure.

### RNA, DNA, and Nanoparticle Design

The activatable nanoparticle systems and split-functional nanoparticles with ssRNA toeholds described herein may comprise DNA, RNA, and hybrids of DNA and RNA. The present invention contemplates a flexible approach to designing interacting nanoparticles for use in the activatable nanoparticle systems described herein. Indeed, any form or type or category of nanoparticle may be used so long as interacting nanoparticles each comprise one or more complementary or cognate duplexes with ssRNA toeholds wherein the duplexes undergo mutual strand reassociation (i.e., swapping of strands as between duplexes) in a manner that is dependent on the initial interaction of the cognate or complementary ssRNA toeholds in each of the interacting nanoparticles (i.e., ssRNA toehold-dependent interaction). Any of the nanoparticles described or contemplated in the inventors' own publicly-available patent applications or publications may be retrofitted with ssRNA toeholds as described herein. Such publications include any of the following, each of which are incorporated herein by reference:

PCT/US2007/013027 (WO2008/039254) (“RNA Nanoparticles and Nanotubes”), filed May 31, 2007, PCT/US2010/038818 (WO 2010/148085) (“RNA Nanoparticles and Nanotubes”), filed Jun. 16, 2010, PCT/US2012/065932 (WO 2013/075132)(“Therapeutic RNA Switches), filed Nov. 19, 2012, PCT/US2012/065945 (WO 2013/075140) (“Auto-Recognizing Therapeutic RNA/DNA Chimeric Nanoparticles”), filed Nov. 19, 2012, PCTUS2013/058492 (WO 2014/039809) (“Co-Transcriptional Assembly of Modified RNA Nanoparticles”), filed Sep. 6, 2013, PCT/US2014/056007 (WO 2015/042101) (“Multifunctional RNA Nanoparticles and Methods of Use”), filed Sep. 17, 2014, and PCT/US2015/029553 (WO 2015/171827) (“Triggering RNA Interference with RNA-DNA and DNA-RNA Nanoparticles”), filed May 6, 2015.

Any of the nanoparticles described in the inventors' own scientific peer-reviewed articles may also be retrofitted with ssRNA toeholds and used in the activatable nanoparticles systems of the invention. The references include:
1: Halman J R, Satterwhite E, Roark B, Chandler M, Viard M, Ivanina A, Bindewald E, Kasprzak W K, Panigaj M, Bui M N, Lu J S, Miller J, Khisamutdinov E F, Shapiro B A, Dobrovolskaia M A, Afonin K A. Functionally-interdependent shape-switching nanoparticles with controllable properties. Nucleic Acids Res. 2017 Jan. 20. pii: gkx008. doi: 10.1093/nar/gkx008. [Epub ahead of print] PubMed PMID: 28108656.
2: Parlea L, Puri A, Kasprzak W, Bindewald E, Zakrevsky P, Satterwhite E, Joseph K, Afonin K A, Shapiro B A. Cellular Delivery of RNA Nanoparticles. ACS Comb Sci. 2016 Sep. 12; 18(9):527-47. doi: 10.1021/acscombsci.6b00073. PubMed PMID: 27509068.
3: Parlea L, Bindewald E, Sharan R, Bartlett N, Moriarty D, Oliver J, Afonin K A, Shapiro B A. Ring Catalog: A resource for designing self-assembling RNA nanostructures. Methods. 2016 Jul. 1; 103:128-37. doi: 10.1016/j.ymeth.2016.04.016. PubMed PMID: 27090005.
4: Afonin K A, Viard M, Tedbury P, Bindewald E, Parlea L, Howington M, Valdman M, Johns-Boehme A, Brainerd C, Freed E O, Shapiro B A. The Use of Minimal RNA Toeholds to Trigger the Activation of Multiple Functionalities. Nano Lett. 2016 Mar. 9; 16(3):1746-53. doi: 10.1021/acs.nanolett.5b04676. PubMed PMID: 26926382.
5: Afonin K A, Viard M, Kagiampakis I, Case C L, Dobrovolskaia M A, Hofmann J, Vrzak A, Kireeva M, Kasprzak W K, KewalRamani V N, Shapiro B A. Triggering of RNA interference with RNA-RNA, RNA-DNA, and DNA-RNA nanoparticles. ACS Nano. 2015 Jan. 27; 9(1):251-9. doi: 10.1021/nn504508s. PubMed PMID: 25521794; PubMed Central PMCID: PMC4310632.
6: Afonin K A, Viard M, Koyfman A Y, Martins A N, Kasprzak W K, Panigaj M, Desai R, Santhanam A, Grabow W W, Jaeger L, Heldman E, Reiser J, Chiu W, Freed E O, Shapiro B A. Multifunctional RNA nanoparticles. Nano Lett. 2014 Oct. 8; 14(10):5662-71. doi: 10.1021/n502385k. PubMed PMID: 25267559; PubMed Central PMCID: PMC4189619.
7: Afonin K A, Kasprzak W K, Bindewald E, Kireeva M, Viard M, Kashlev M, Shapiro B A. In silico design and enzymatic synthesis offunctional RNA nanoparticles. Acc Chem Res. 2014 Jun. 17; 47(6):1731-41. doi: 10.1021/ar400329z. PubMed PMID: 24758371; PubMed Central PMCID: PMC4066900.
8: Afonin K A, Desai R, Viard M, Kireeva M L, Bindewald E, Case C L, Maciag A E, Kasprzak W K, Kim T, Sappe A, Stepler M, Kewalramani V N, Kashlev M, Blumenthal R, Shapiro B A. Co-transcriptional production of RNA-DNA hybrids for simultaneous release of multiple split functionalities. Nucleic Acids Res. 2014 February; 42(3):2085-97. doi: 10.1093/nar/gkt1001. PubMed PMID: 24194608; PubMed Central PMCID: PMC3919563.
9: Afonin K A, Viard M, Martins A N, Lockett S J, Maciag A E, Freed E O, Heldman E, Jaeger L, Blumenthal R, Shapiro B A. Activation of different split functionalities on re-association of RNA-DNA hybrids. Nat Nanotechnol. 2013 April; 8(4):296-304. doi: 10.1038/nnano.2013.44. PubMed PMID: 23542902; PubMed Central PMCID: PMC3618561.
10: Afonin K A, Kireeva M, Grabow W W, Kashlev M, Jaeger L, Shapiro B A. Co-transcriptional assembly of chemically modified RNA nanoparticles functionalized with siRNAs. Nano Lett. 2012 Oct. 10; 12(10):5192-5. doi: 10.1021/n302302e. PubMed PMID: 23016824; PubMed Central PMCID: PMC3498980.
11: Afonin K A, Grabow W W, Walker F M, Bindewald E, Dobrovolskaia M A, Shapiro B A, Jaeger L. Design and self-assembly of siRNA-functionalized RNA nanoparticles for use in automated nanomedicine. Nat Protoc. 2011 Dec. 1; 6(12):2022-34. Doi 10.1038/nprot.2011.418. PubMed PMID: 22134126; PubMed Central PMCID: PMC3498981.
12: Shapiro B A, Bindewald E, Kasprzak W, Yingling Y. Protocols for the in silico design of RNA nanostructures. Methods Mol Biol. 2008; 474:93-115. doi: 10.1007/978-1-59745-480-3_7. Review. PubMed PMID: 19031063.
13: Bindewald E, Grunewald C, Boyle B, O'Connor M, Shapiro B A. Computational strategies for the automated design of RNA nanoscale structures from building blocks using NanoTiler. J Mol Graph Model. 2008 October; 27(3):299-308. Doi: 10.1016/j.jmgm.2008.05.004. PubMed PMID: 18838281; PubMed Central PMCID: PMC3744370.
14: Yingling Y G, Shapiro B A. Computational design of an RNA hexagonal nanoring and an RNA nanotube. Nano Lett. 2007 August; 7(8):2328-34. PubMed PMID: 17616164.

All of these references are incorporated herein by reference in their entireties.

RNA has a number of advantages for nanostructure design. Nanoparticle structures provide a size range that is large enough to avoid the problem of expulsion from the cell, but are small enough to avoid the problems of cell delivery often encountered with larger particles. RNA is the only biopolymer that can carry genetic information and has catalytic properties. RNA can naturally fold into complex motifs, and RNA motifs are capable of self-assembly. RNA has a natural functionality, for instance RNA can function as ribozymes or riboswitches. Further, RNA is advantageous in eliciting a very low immune response. Moreover, the construction of RNA into ordered, patterned superstuctures has a number of desirable characteristics, including the ability to self-assemble in precisely defined ways, the ability to undergo editing and replication, the ability to undergo controlled disassembly. RNA has versatility in function and structure. Functionally, RNA is the only biopolymer that can carry genetic information and that possesses catalytic properties. Structurally, RNA has predictable intra and intermolecular interactions with well-known structural geometry. The RNA strands that consist of adenine (A), guanine (G), cytosine (C), and uridine (U) can naturally, or can be programmed, to self-assemble via complementary base pairing. The helical region of RNA has a well-known nanometer scale structural geometry of 2.86 nm per helical turn with 11 base pairs and a 2.3 nm diameter. The self-assembly of RNA into complex structures can be facilitated via complementary base pairing or inter- and intra-molecular interactions of the different single stranded regions in the RNA, including internal bulges and loop motifs, and single-stranded overhangs or “sticky-ends”. In addition to Watson-Crick base pairing, A, G, C and T can also pair with other, unconventional bases (i.e. non-canonical base-pairing).

The methods of the invention can be used to assemble RNA NPs composed of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 112, 113, 114, 115, 116, 117, 118, 119, 120, 121, 122, 123, 124, 125 or more distinct RNA strands.

### RNA and DNA Synthesis

The activatable nanoparticles of the invention comprise DNA and RNA molecules which may be made using any suitable and known means. For example, RNA and DNA molecules used to make the nanoparticles of the invention can be produced recombinantly or synthetically by methods that are routine for one of skill in the art. For example, synthetic RNA molecules can be made as described in US Patent Application Publication No.: 20020161219, or U.S. Pat. Nos. 6,469,158, 5,466,586, 5,281,781, or 6,787,305. Fluorescently labeled RNA molecules were purchased from Integrated DNA Technologies, Inc.

### RNA Self-Assembly

The activatable nanoparticles of the invention may rely on the characteristics of RNA self-assembly to form nanoparticles. Small RNA structural motifs can code the precise topology of large molecular architectures. It has been shown that RNA structural motifs participate in a predictable manner to stabilize, position and pack RNA helices without the need of proteins (Chworos A et al., Science 306:2068-2072.2004). RNAI and RNAII are loop structures that interact in what is called a ‘kiss’ or ‘kissing’ complex (Lee et al., Structure 6:993-1005.1998). This contact facilitates the pairing of the RNAI and RNAII loops, until the two RNAs form a duplex. As such, the “kissing” interaction between RNAI and RNAII is one means of self-assembly between the RNA building blocks. This self-assembly strategy relies on the particular geometry of bended kissing complexes. The nanorings formed, can be functionalized with siRNA sequences and are capable of being processed by dicer,can be used as siRNA delivery systems (Grabow et al., Nano letters 11:878-887.2011) has potential for developing siRNAs delivery agents.

The self-assembly of nanoparticles from RNA involves cooperative interaction of individual RNA molecules that spontaneously assemble in a predefined manner to form a larger two- or three-dimensional structure. Within the realm of self-assembly two main categories have been described: template and non-template (Lee et al. J Nanosci Nanotechnol. 2005 December; 5(12):1964-82). Template assembly involves interaction of RNA molecules under the influence of specific external sequence, forces, or spatial constraints such as RNA transcription, hybridization, replication, annealing, molding, or replicas. In contrast, non-template assembly involves formation of a larger structure by individual components without the influence of external forces. Examples of non-template assembly are ligation, chemical conjugation, covalent linkage, and loop/loop interaction of RNA, especially the formation of RNA multimeric complexes (Lee et al. 2005, as above).

Previously, RNA has been demonstrated to assemble into nanoparticles of various shapes and sizes. The first RNA nanoparticles were generated using loop-receptor interfaces to form dimeric nanoparticles. The assembly of this H-shaped nanoparticle was mediated by GAAA/Hnt receptor interaction, which is a highly recurrent motif found in group I and group II introns and other ribozymes and riboswitches. This interaction was further used to generate oriented filaments by combining multiple loop-receptor interactions with a four-way junction motif One of the first examples of RNA nanoparticles that incorporate multiple RNA motifs within its context is the tectosquare, which is composed of four artificial RNA building blocks called tectoRNAs that self-assemble through specific, non-covalent loop-loop interactions called kissing loops (KL) found at the end of each stem. These tectoRNAs were further programmed to self-assemble into complex arrays via 3′ sticky tails with controllable topology, directionality and geometry. The first example of a therapeutic RNA nanoparticle was designed from phi-29-encoded packaging motor (pRNA), a natural RNA motif found in bacteriophages. The pRNA dimers were reengineered for targeted delivery of ribozymes to attack the hepatitis B virus by specifically cleaving the virus's poly-A signal. In a subsequent study, the pRNA trimers were functionalized with cell receptor-binding RNA aptamers and were used to deliver siRNAs that target a specific gene for silencing and thus enabling apoptosis in cancer cells.

### Nanoparticle Characteristics and Manufacture

As used herein, the terms “nanoparticle” and “nanoscaffold” are interchangeable. The terms “nanotube,” “nanoring,” and “nanocube” may refer to specific three dimensional forms of the nanoparticles, all of which are contemplated herein. In preferred embodiments, the invention relates to RNA-based nanoparticles (e.g., nanorings or nanocubes) comprising a core structure (e.g., a ring, square, or cube) of DNA or RNA which is functionalized with one or more DNA/RNA hybrid “arms” attached thereto, wherein the RNA component of the hybrid arms comprises a single stranded RNA toehold sequence.

The core structure of any of the activatable nanoparticles of the invention can be in the shape of any two-dimensional shape, such as a ring (e.g., a hexaganol ring), a square, a sheet, or the like. The core structure can also be in the shape of any three-dimensional structure, such as, a cube, a prism, or a tube. In preferred embodiments, the core structure is formed of self-assembly RNA oligonucleotides. Methods for forming various core structures (e.g., nanorings, nanotubes, or nanocubes) have been described in the inventors' prior applications, for example in PCT/US2007/013027 (“RNA Nanoparticles and Nanotubes”), filed May 31, 2007, PCT/US2010/038818 (“RNA Nanoparticles and Nanotubes”), filed Jun. 16, 2010, PCT/US2012/065932 (“Therapeutic RNA Switches), filed Nov. 19, 2012, PCT/US2012/065945 (“Auto-Recognizing Therapeutic RNA/DNA Chimeric Nanoparticles”), filed Nov. 19, 2012, PCTUS2013/058492 (“Co-Transcriptional Assembly of Modified RNA Nanoparticles”), filed Sep. 6, 2013, PCT/US2014/056007 (“Multifunctional RNA Nanoparticles and Methods of Use”), filed Sep. 17, 2014, and PCT/US2015/029553 (“Triggering RNA Interference with RNA-DNA and DNA-RNA Nanoparticles”), each of which is incorporated herein by reference.

In certain embodiments, the core portion of the RNA-based nanoparticles of the invention may be modified by attaching one or more DNA/RNA hybrid “arm” that comprises a latent functionality that forms only upon reassociating the DNA/RNA hybrid “arm” with a cognate free DNA/RNA hybrid molecule to form a functional RNA/RNA duplex. The RNA/RNA duplex could be, for example, an siRNA molecule for triggering RNA interference of a specific target. In addition, the RNA/RNA duplex could comprise FRET pairs, which are activated to produce fluorescence only upon the formation of the RNA/RNA duplex. The hybrid arms preferably comprise an ssRNA toehold sequence for facilitating interaction between two or more cognate nanoparticles described herein.

Methods and sequences for producing a RNA-based nanoparticle having a RNA or DNA core and functionalized with a DNA/RNA hybrid “arm” can be found described, for example, in PCT/US2014/056007 (“Multifunctional RNA Nanoparticles and Methods of Use”), filed Sep. 17, 2014, and PCT/US2015/029553 (“Triggering RNA Interference with RNA-DNA and DNA-RNA Nanoparticles”), which are incorporated herein by reference.

In certain preferred embodiments, the RNA component of the DNA/RNA hybrid “arm” comprises a single stranded RNA “toehold” sequence that extends from the end the hybrid arm by at least 1 additional ribonucleotide. In certain embodiments, the RNA toehold sequences (i.e., the portion of the RNA component of the DNA/RNA hybrid that is single stranded) are less than 4 nucleotides in length. In other embodiments, the RNA toehold sequences are 4 or more nucleotides in length. In still other embodiments, the RNA toehold sequences are between 4 and 6 nucleotides, or between 5 and 7 nucleotides, or between 6 and 8 nucleotides, or between 7 and 9 nucleotides, or between 8 and 10 nucleotides, or between 9 and 11 nucleotides, or between 10 and 12 nucleotides, or between 11 and 13 nucleotides, or between 12 and 14 nucleotides, or between 13 and 15 nucleotides, or between 14 and 16 nucleotides, or more than 16 nucleotides. In other embodiments, the single stranded RNA toehold sequences are 3, 4, 5, 6, 7, 8, 9, 10, 11, 12, 13, 14, 15, 16, 17, 18, 19, or 20 nucleotides in length.

Using natural or artificially selected RNA motifs and modules, RNA molecules can be programmed to form a wide variety of compact and stable artificial three-dimensional nanostructures (called RNA NPs; Afonin et al. Accounts of Chemical Research 2014, dx.doi.org/10.1021/ar400329z; Afonin et al. Nat Nanotechnol 2010, 5, (9), 676-82; Severcan et al. Nat Chem 2010, 2, (9), 772-9; Grabow et al. Nano Lett 2011, 11, (2), 878-87; Guo et al., M. Mol Cell 1998, 2, (1), 149-55) suitable for the broad range of clinical and nanotechnological applications (Afonin et al. Accounts of Chemical Research 2014, dx.doi.org/10.1021/ar400329z; Afonin et al. Nat Protoc 2011, 6, (12), 2022-34; Guo, P. Nat Nanotechnol 2010, 5, (12), 833-42; Shukla et al. ACSNano 2011, 5, (5), 3405-3418; Shu et al. Rna 2013, 19, (6), 767-77; Koyfman et al. JAm Chem Soc 2005, 127, (34), 11886-7; Shu et al. Adv Drug Deliv Rev 2014, 66C, 74-89; Khisamutdinov et al. ACS Nano 2014; Hao et al. Nat Commun 2014, 5, 3890; Ohno et al. Nat Nanotechnol 2011, 6, (2), 116-20; Osada et al. ACS Nano 2014; Haque et al. Nano Today 2012, 7, (4), 245-257; Tarapore et al. Mol Ther 2011, 19, (2), 386-94). Therapeutic nucleic acids, proteins, or small molecules can be individually attached using different techniques (Shu et al. Adv Drug Deliv Rev 2014, 66C, 74-89) to programmable RNA toeholds entering the composition of R/DNA NP. The assembly of the monomers will bring the desired functionalities together, thus providing precise control over their topology, composition, and modularity. The use of functional R/DNA NP with ssRNA toeholds in vivo will guarantee higher concentration and desired stoichiometry of therapeutic moieties locally.

Herein, new multifunctional R/DNA NPs built based on previously designed RNA nanorings (Grabow et al. Nano Lett 2011, 11, (2), 878-87; Yingling and Shapiro. Nano Lett 2007, 7, (8), 2328-34) were identified, with the inventions illustrating how this system can be used to address several present challenges associated with RNA NPs including functionalization with different classes of molecules such as multiple siRNAs, aptamers, proteins, and small molecules. Detailed characterization of the resulting functional RNA NPs in vitro (by native-PAGE, DLS, cryo-EM, and fluorescent studies), in various cell cultures and in vivo was demonstrated.

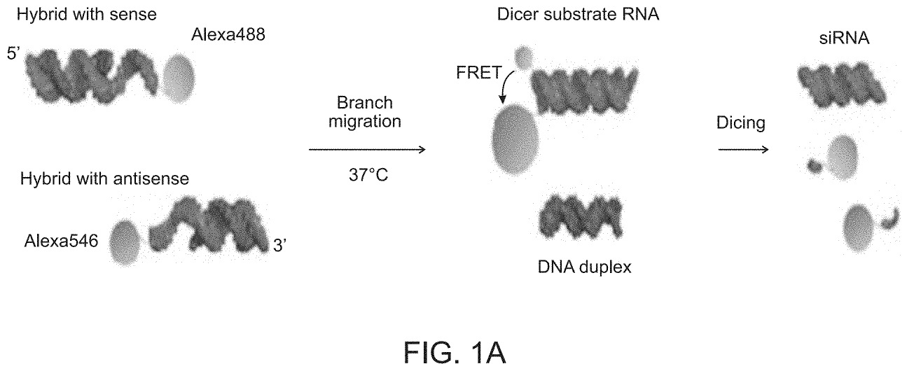

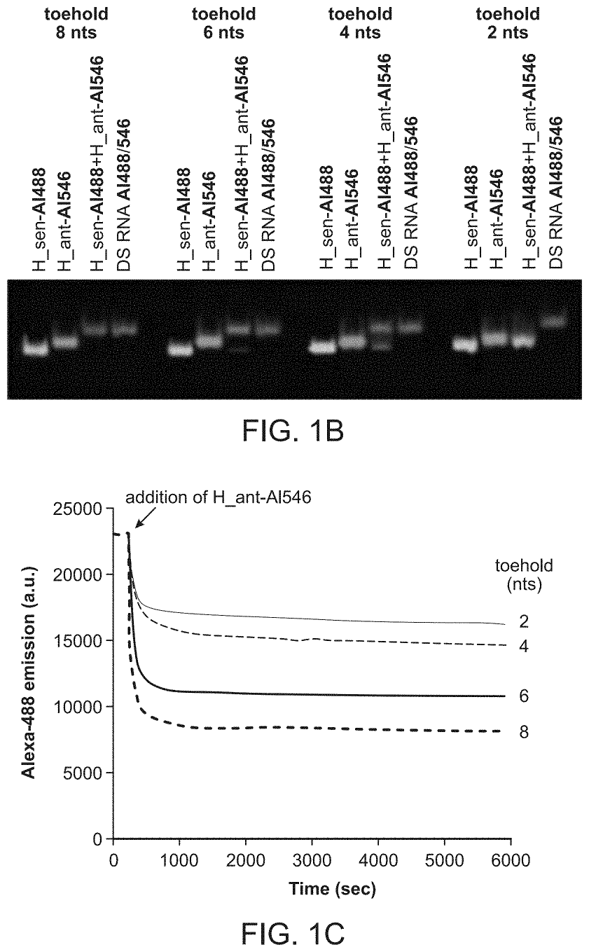

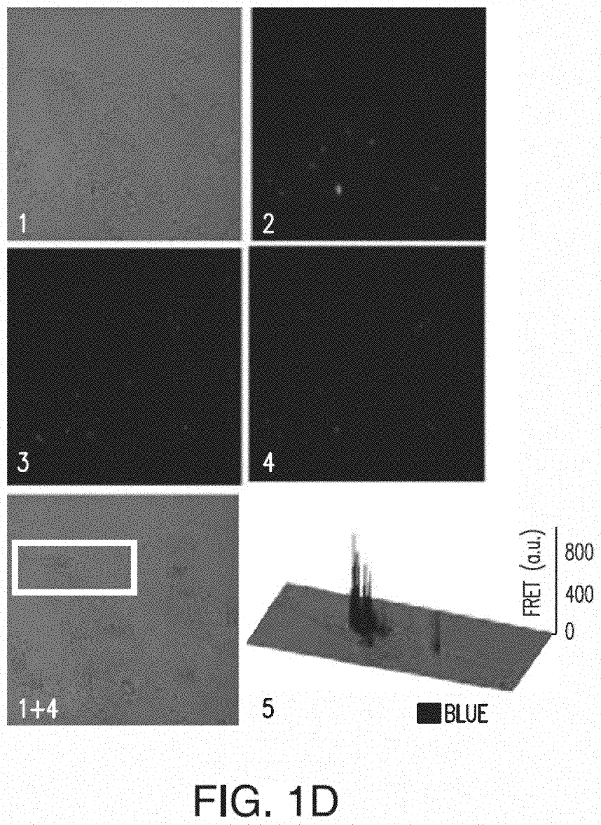

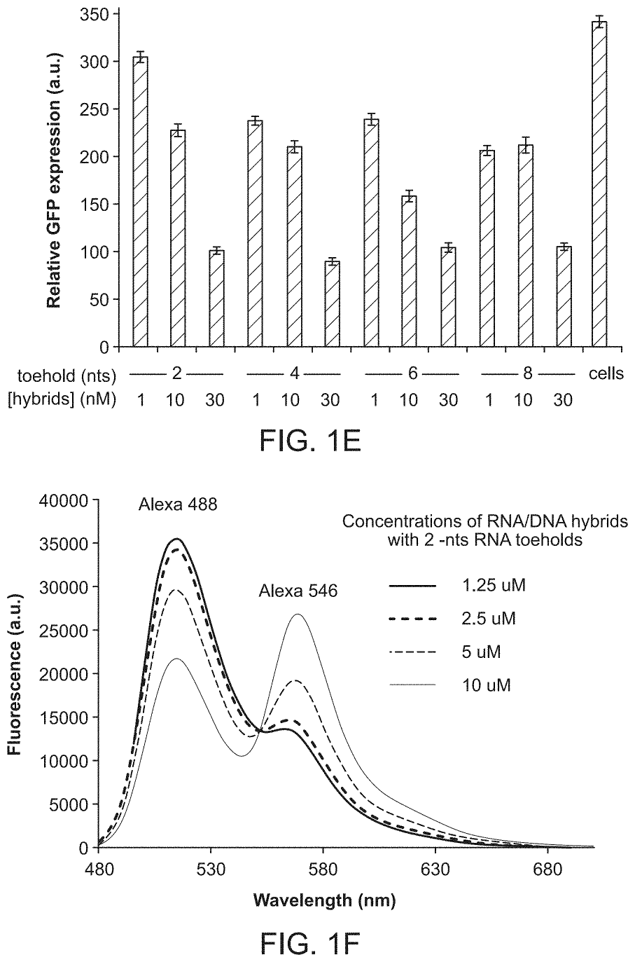

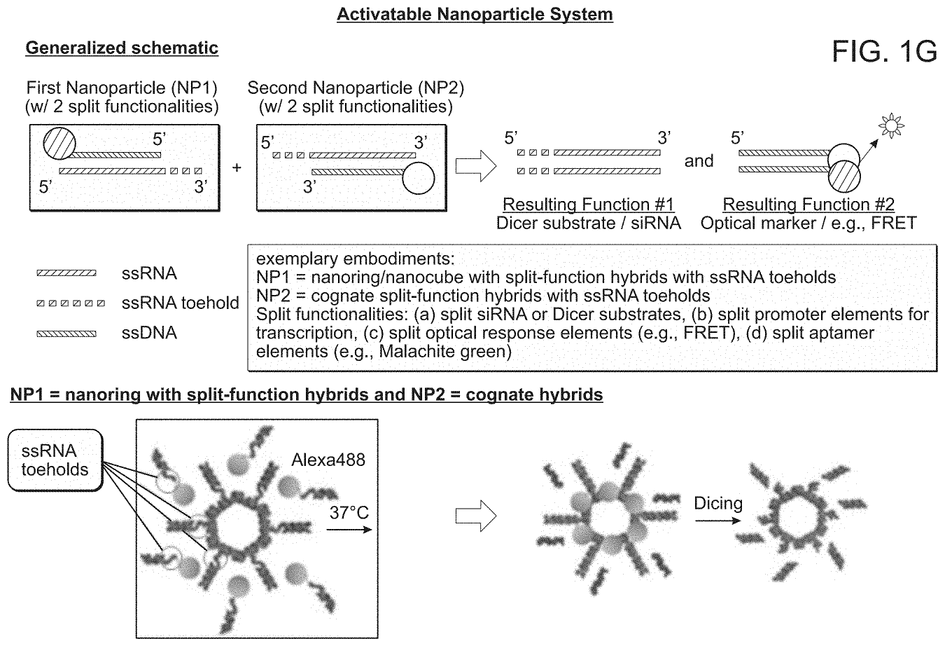

As the proof of concept, the function of Dicer Substrate RNA (DS RNA) was split between two R/DNA hybrids. DS RNA was designed to downregulate the production of green fluorescent protein (GFP) (Rose et al. Nucleic acids research 2005, 33, 4140-4156) that is stably expressed in model human breast cancer cells (MDA-MB231/GFP). The use of DS RNAs (as opposed to siRNAs) is required to ensure that once inside the cells, the individual hybrids will not be active in the RNAi pathway (Afonin et al. Nucleic acids research 2014, 42, 2085-2097.). GFP DS RNAs were split between two RNA-DNA hybrids with the DNA strands being 8-, 6-, 4-, and 2-nts shorter than their corresponding complementary RNAs, thus, providing the ssRNA toeholds for further re-association. A scheme explaining the re-association of new hybrids studied in this work is shown in FIG. 1A.

The incorporation of RNA functionalities such as Dicer Substrate (DS) RNAs (Rose et al. Nucleic Acids Res 2005, 33, (13), 4140-56) into the nanoscaffolds presented difficulties in terms of solid state chemical synthesis as RNA components generally cannot exceed ˜60 nucleotides in length. This problem was addressed by using small SS RNA toeholds with split functionalities in the cognate hybrids.

Lastly, it has been established herein how the therapeutic functionality of the nanoring can be triggered through the incorporation of multiple agents/functionalities at ssRNA toeholds of RNA-DNA hybrids. This newly developed technique (Afonin et al. Nat Nanotechnol 2013, 8, (4), 296-304; Afonin et al. Acc Chem Res 2014) involves splitting the different functionalities between cognate RNA-DNA hybrids with further conditional intracellular activation of these functionalities.

In addition to functionalization with multiple different short interfering RNAs for combinatorial RNA interference (e.g. against multiple HIV-1 genes), nanorings of the invention also allow simultaneous embedment of fluorescent dyes, proteins, as well as recently developed RNA-DNA hybrids aimed to conditionally activate multiple split functionalities inside cells.

### Design

The general approach used to create RNA nano-particles and nano-materials is to take known RNA structures, cut them into the building blocks, and reengineer single-stranded loops and regions to facilitate the desired self-assembly. The self-assembly of all the above discussed RNA building blocks into nanostructures is mediated by the complementarity of hairpin loops and loop receptors that form non-covalent RNA-RNA interactions. For precise assembly of the RNA building blocks, each of the corresponding complementary loop-loop interactions are uniquely reengineered.

Two main experimental approaches can be used for programmable self-assembly of nucleic acids nanostructures (Jaeger, L.; Chworos, A. Curr Opin Struct Biol 2006, 16, (4), 531-43). The first is a single-step assembly, which is commonly used for DNA nanostructures (Chelyapov, N.; Brun, Y.; Gopalkrishnan, M.; Reishus, D.; Shaw, B.; Adleman, L. J Am Chem Soc 2004, 126, (43), 13924-5; Mathieu, F.; Liao, S.; Kopatsch, J.; Wang, T.; Mao, C.; Seeman, N. C. Nano Lett 2005, 5, (4), 661-5.). The second is a stepwise assembly, which has been commonly described for RNA nanostructures (Chworos, A.; Severcan, I.; Koyfman, A. Y.; Weinkam, P.; Oroudjev, E.; Hansma, H. G.; Jaeger, L. Science 2004, 306, (5704), 2068-72). In the single-step assembly approach, all molecules are mixed together followed by the slow cool annealing procedure. This is only possible if the target building block structure is the one that has the highest number of Watson-Crick base pairs and is therefore the most stable. However, it is understood that thermodynamic stability of different shapes of nanoparticles is also an important consideration, at times more so than Watson base pairing. This approach is, thus, based on the preferential folding of the building blocks at higher temperatures followed by the self-assembly of these building blocks through weaker interactions into final nanostructures at lower temperatures. However, usually there are many other possible structures that are only slightly less stable. In this case, the stepwise approach can be used where the building blocks are separately formed in the first step are then mixed together in the presence of high magnesium (Mg++) concentration to form a final nanostructure. This approach is more time consuming and the melting temperatures of the building blocks and the final nanostructure should be well separated.

A number of RNA motifs are available as building blocks, including but not limited to RNA I and/or RNA II motifs, kissing loops, RNA I inverse (RNA Ii) and/or RNA II inverse (RNA IIi) motifs. As used herein, the term “motif” in reference to a nanoparticle is meant to refer to a double-stranded or single-stranded ribonucleic acid or analog thereof. Individual motifs are joined together into larger particles by attachment to each other. Attachment can occur by non-covalent linking. Numerous high-resolution RNA structures determined by NMR or X-ray crystallography can be separated into building blocks for design of new RNA nanoparticles and nanomaterials. U.S. application Ser. No. 13/378,985, incorporated by reference in its entirety herein, describes methods of making RNA nanoparticles.

The RNA NPs comprising one or more functionalities according to the invention can be in the shape of a ring, in the shape of a square or in the shape of a triangle; however it is to be understood that other geometries are possible. In certain embodiments, there is a positive relationship between the stability of RNA assemblies and the complexity of the tertiary structures that define the assembly.

### R/DNA Hybrid Duplexes

In various embodiments, e.g., FIG. 1G, the interacting nanoparticles described herein comprise one or more functional hybrid duplex “arms.” It is preferred the arms comprise a ssRNA toehold sequence to catalyze or initiate strand reassociation/swapping between the interacting nanoparticles comprising the hybrid duplexes. In certain embodiments, the present invention splits the functionality, e.g., Dicer substrates siRNA duplexes, into two R/DNA hybrids, which upon simultaneous presence inside the same diseased cell will recognize each other through toehold interaction and re-associate releasing active siRNAs. This approach will overcome several challenges associated with the clinical delivery of RNAi, such as intravascular degradation (will be reduced for R/DNA hybrids), tissue specificity (DNA chemistry is more parsimonious than RNA and amenable to chemical modifications with different features for targeting or delivery), pharmacodynamics (fluorescent tags can be activated upon R/DNA hybrid re-association assisting in Forster resonance energy transfer (FRET) imaging of delivery and response). R/DNA hybrids are described in PCT/US2012/065945 and incorporated by reference in its entirety herein.

Using RNA interference (RNAi) as a therapeutic agent it is routinely possible to knock down the expression of target genes in diseased cells. In certain embodiments, the ability of the hybrids with ssRNA toeholds to enter and re-associate inside mammalian cells was assessed (FIG. 1D). The invention features a method for siRNA release where cognate hybrids are co-delivered to the cell either on the same or on two different days. The invention provides for nucleic acids based “smart” nanoparticles for biomedical applications.

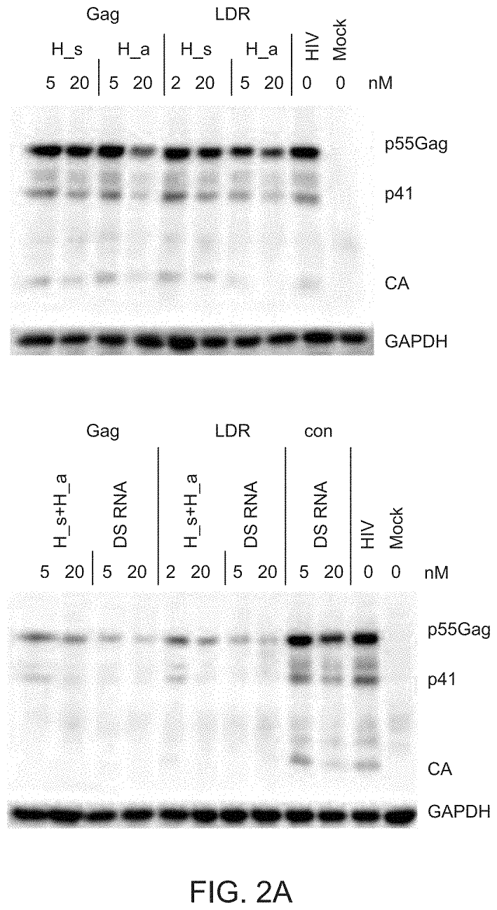

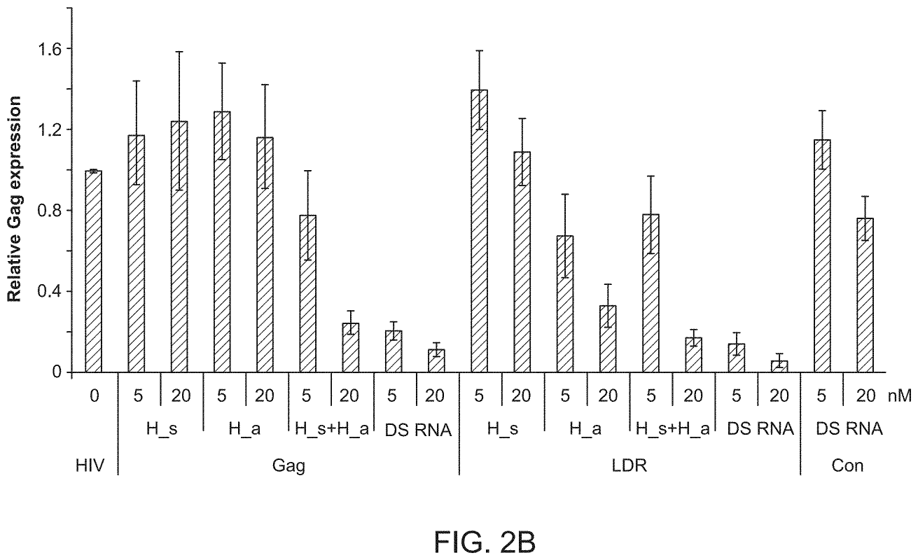

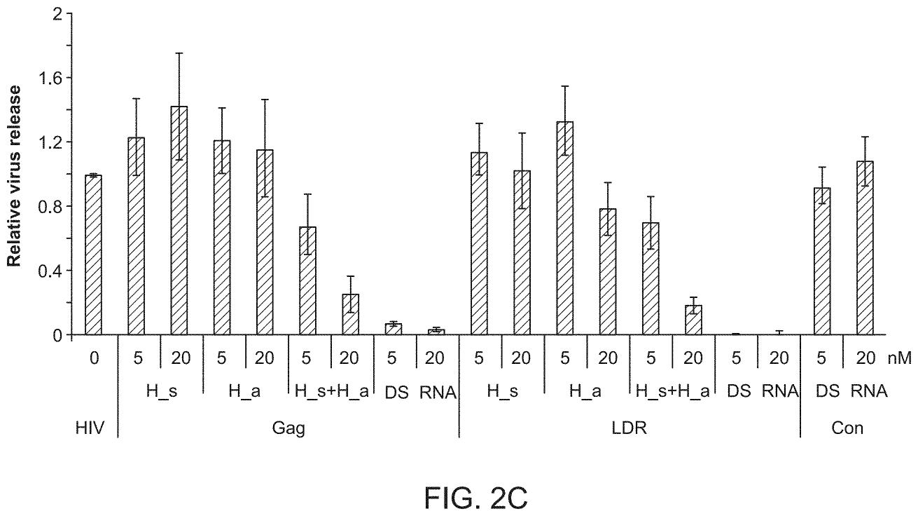

To demonstrate the generality and the therapeutic potential of this approach, two additional sets of hybrids were designed against the full-length genomic HIV-1 RNA that also serves as the mRNA coding for the viral structural proteins and enzymes (Berkhout et al. Antiviral research 2011, 92, 7-14; Liu et al., Molecular therapy: the journal of the American Society of Gene Therapy 2009, 17, 1712-1723; Low et al. Molecular therapy: the journal of the American Society of Gene Therapy 2012, 20, 820-828; Olivier et al. Molecular therapy: the journal of the American Society of Gene Therapy 2008, 16, 557-564). Gag and LDR hybrids targeted the sequences coding the capsid domain and the amino terminus of the matrix domain of Gag respectively (FIG. 2A-2B). Both hybrids were designed to have 8-nt ssRNA toeholds. Transfection of the individual hybrids showed no significant effect on relative virus release. However, the co-transfection of cognate hybrids reduced the production of virus significantly (FIG. 2C).

In certain embodiments, the design rationale of R/DNA hybrids is the following: functional Dicer substrate siRNAs are split between ssRNA toeholds of two cognate R/DNA hybrids preventing them from being diced and thus, making them non-functional.

ssRNA Toehold Interaction

The RNA-based nanoparticles, e.g., RNA/DNA hybrid particles, comprise in certain preferred embodiments single strand RNA toehold sequences, particularly to enhance the interaction and reassociation of RNA/RNA strand pairs as between an RNA/DNA hybrid particle and a cognate RNA/DNA hybrid molecule.

Without being bound by theory, hybridization of the invading strand is initiated at a short single stranded “toehold” domain attached to one end of the substrate, leading to a branch migration reaction that displaces the target strand from the substrate.

The term “single strand RNA toehold” refers to nucleation site of a domain comprising an RNA sequence designed to initiate hybridization of the domain with a complementary RNA sequence. The secondary structure of a nanoparticle may be such that the toehold is exposed or sequestered. For example, in some embodiments, the secondary structure of the toehold is such that the toehold is available to hybridize to a complementary nucleic acid (the toehold is “exposed,” or “accessible”), and in other embodiments, the secondary structure of the toehold is such that the toehold is not available to hybridize to a complementary nucleic acid (the toehold is “sequestered,” or “inaccessible”). If the toehold is sequestered or otherwise unavailable, the toehold can be made available by some event such as, for example, the opening of the hairpin of which it is a part of. When exposed, a toehold is configured such that a complementary nucleic acid sequence can nucleate at the toehold.

A scheme of re-association for the hybrids is described in PCT/US2012/065945, which is incorporated by reference in its entirety herein. The complementary single-stranded unzipped toeholds in R/DNA hybrids are designed using Mfold (Zuker, M, Nucleic Acids Res 31, 3406-3415 (2003)) to avoid any stable secondary structures. In order to exceed a melting temperature (Tm) of 37° C., the minimal length of the unzipped toeholds with GC content >60 should be at least 12 nucleotides (nts). The Tm for designed single stranded toeholds is estimated to be 40° C. using the Wallace rule (Wallace, R. B. et al., Nucleic Acids Res 6, 3543-3557 (1979)).

### Computational Prediction

In certain embodiments, an advanced algorithm has been used for the computational predictions of R/DNA hybrid re-associations and RNA secondary structures. In-silico secondary structure predictions are required for the further advancement of RNA based nanoparticles by understanding and utilizing the interactions of RNA strands with non-nested base pairing. The novel computational approach (which can be referred to herein in one embodiment as “HyperFold”) is capable of predicting base pairing of RNA/RNA, DNA/DNA and RNA/DNA hybrid interactions.

A variety of secondary structure prediction programs for multiple RNA strands have been already reported in literature (Lorenz et al, Algorithms Mol Biol 2011, 6, 26; Cao et al, RNA 2014, 20, 835; Bernhart et al, Algorithms Mol Biol 2006, 1, 3; Bindewald et al, ACS Nano 2011, 5, 9542; Andronescu et al, J Mol Biol 2005, 345, 987). The NUPACK software is in addition to RNA/RNA interactions also capable of predicting DNA/DNA interactions (Dirks et al, J Comput Chem 2003, 24, 1664; Zadeh et al, J Comput Chem 2010, 32, 170). Secondary structure predictions of RNA/DNA hybrid duplexes have been made available via the RNA Vienna package (Lorenz et al, Algorithms Mol Biol 2011, 6, 26).

The software described here is unique in the sense that it allows for secondary structure predictions of multiple nucleic acids strands simultaneously taking into account possible RNA/RNA, RNA/DNA and DNA/DNA interactions which may include pseudoknots. RNA structures with non-nested base pairings (in other words secondary structures whose circular diagram representations contain “crossing arcs”) are referred to as pseudoknotted (Bindewald et al, ACS Nano 2011, 5, 9542). The presented prediction approach does consider base pairs that are non-nested with respect to each other even if the involved nucleotides involve more than one nucleotide strand.

HyperFold performs competitively compared to other published folding algorithms. A large variety of RNA/DNA hybrid re-association experiments were performed for different concentrations, RNA toehold lengths, and G+C content and it was observed that tendencies for re-association correspond well to computational predictions.

The computational structure prediction approach was also used to predict equilibrium concentrations of complexes consisting of RNA/DNA hybrid sequences. For all toehold lengths and concentrations, it was found computationally that the re-associated RNA and DNA duplexes correspond to the lowest free energy conformation. In other words, the computational results suggest, that experimentally observed dramatic differences in re-association are due to kinetic effects. This is also confirmed by experimental results where re-association is observed for RNA/DNA hybrids without toeholds provided they have high concentration. The computationally estimated values when plotted together with the experimentally determined re-association rates then it corresponds to different concentrations and toehold lengths, follows fairly well a sigmoidal curve as one would expect for a chemical system that contains a kinetic barrier.

### Conjugation to Nanoparticles

The nanoparticles described herein may also be configured for delivering agents. For example, nanoparticles can be used to deliver one or more agents that are selected from one or more of the group consisting of: siRNAs, RNA or DNA aptamers, fluorescent dyes, small molecules, RNA-DNA hybrids with split functionalities, split lipase, split GFP, proteins, therapeutic agents and imaging agents.

The compositions of the present invention have therapeutic uses. Any number of diseases or disorders can be treated by the compositions of the present invention and may be limited, in fact, only by the agent or agents that can be loaded in the inside of the nanoparticle or conjugated to the outside.

For example, RNA NPs can be engineered to carry multiple siRNAs against different disease targets. In one exemplary embodiment, six different siRNAs against different parts of the HIV-1 genome can be used for combinatorial RNAi therapy. The invention is not limited HIV, or to any disease or group of diseases, but is rather defined by the siRNAs that can be used to treat particular diseases. This concept of targeting a specific pathway upon the presence of a particular RNA in the cytoplasm can be applied to cancer (including cancer stem cells) or RNA viruses in general (e.g. Flaviviruses, Alphaviruses). HAART therapy as it currently exists, can successfully suppress virus replication within the human host. With this approach, however, it is currently not possible to eradicate the HIV virus from an infected patient because approved HIV drugs act as virus suppressors and do not kill human cells that are infected by the virus. The present invention can also lead to a novel anti-viral drug that has the unique feature of selectively killing HIV infected cells using appropriate aptamers, for cell targeting, that are associated with RNA NPs containing specific siRNAs or RNA/DNA siRNA hybrids. The guide strands are designed to be an antisense to human apoptosis inhibitor genes (BCL-2, FLIP, STAT3, XIAP, SURVIVIN, etc). Thus, the activation of RNAi (RNA interference pathway) will result in apoptosis of the HIV-infected cell. In addition, in a more general sense, the siRNA targets may include cancer related genes, for example, but not limited to, the hypoxia pathway: Hiflalpha, VEGF; DNA repair pathway: PARP; microRNAS: miR21, miR7, mIR128a, mIR210; cancer stem cells: genes in NOTCH, HEDGEHOG, PTEN, WNT, TGFbeta pathways; immune modulation: Interleukin (IL-6, IL-10) and genes in the JAK/STAT, SMAD, TNFalpha. In principle the concept can be expanded to include any genetically related diseases.

Exemplary potential applications of multi-functional nanoparticles of the invention in which 2, 3, 4, or more agents are coupled to a nanoparticle include using one or more agents to target a macromolecular structure or a cell and using the second one to alter the function/properties of the macromolecule or cell, e.g., using a protein to target a cell and using a toxin or cell death protein to kill the targeted cell, using an siRNA to silence genes, or using a fluorescent particle for visualization, or using a chemical or protein to target a protein within a complex and another one to alter the function of a different component of the complex.

In certain embodiments, the nanoparticle comprises one or more agents. In further preferred embodiments, the agent can be conjugated to the nanoparticle. Conjugated can be understood as attached, linked, mixed, or otherwise present on or in a magnetoliposome. For example, an agent can be conjugated by covalent or ionic linkage, by use of a chelate or other linker moiety. As used herein, conjugation of an agent to a nanoparticle does not disrupt the desired activity of the agent.

The agent can comprise any material or compound or composition or agent for in vivo or in vitro use for imaging, diagnostic or therapeutic treatment that can be enclosed in the inside the nanoparticle or can be conjugated with the nanoparticle without appreciably disturbing the physical integrity of the nanoparticle. A nanoparticle can comprise one or more agents of one or more types. For example, a nanoparticle can comprise a therapeutic agent, and the targeting of the agent can be followed by further conjugation with an imaging agent. Similarly, cocktails of therapeutic agents are typically used in the treatment of cancer. A nanoparticle can comprise more than one type of therapeutic agent.

Examples of agents include inhibitory nucleic acids, including but not limited to siRNAs, RNA or DNA aptamers, fluorescent dyes, small molecules, RNA-DNA hybrids with split functionalities, split lipase, split GFP, proteins, therapeutic agents and imaging agents (for example gadolinium, manganese, chromium, or iron).

In certain embodiments, the NP molecules described herein operate by forming inhibitory nucleic acid molecules once in target cells. Such inhibitory nucleic acids include single and double stranded nucleic acid molecules (e.g., DNA, RNA, and analogs thereof) that bind a nucleic acid molecule that encodes target RNA (e.g., antisense oligonucleotide molecules, siRNA, shRNA) as well as nucleic acid molecules that bind directly to a target polypeptide to modulate its biological activity (e.g., aptamers).

Catalytic RNA molecules or ribozymes that include an antisense target RNA sequence of the present disclosure can be used to inhibit expression of target RNAs in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference

The disclosure also features a catalytic RNA molecule that includes, in the binding arm, an antisense RNA having between eight and nineteen consecutive nucleobases. In preferred embodiments of this disclosure, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the disclosure and those skilled in the art will recognize that all that is important in an enzymatic nucleic acid molecule of this disclosure is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

By “siRNA” is meant a double stranded RNA. Optimally, an siRNA is 18, 19, 20, 21, 22, 23, 24 or more nucleotides in length and has a 2 base overhang at its 3′ end. It is understood that the term “siRNA’ includes both diceable and non-diceable siRNAs. These dsRNAs can be introduced to an individual cell or to a whole animal; for example, they may be introduced systemically via the bloodstream. Such siRNAs are used to downregulate mRNA levels or promoter activity. Functional siRNAs can be released by Dicer nuclease. Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39, 2002).

Given the sequence of a target gene, siRNAs may be designed to inactivate that gene. Such siRNAs, for example, could be administered directly to an affected tissue, or administered systemically. The nucleic acid sequence of an Parl gene can be used to design small interfering RNAs (siRNAs). The 21 to 25 nucleotide siRNAs may be used, for example, as therapeutics to inhibit disease related genes.

The inhibitory nucleic acid molecules of the present disclosure may be employed as double-stranded RNAs for RNA interference (RNAi)-mediated knock-down of target RNA expression. In therapeutic embodiments, the target RNA is a disease related gene. For example, in a non-limiting embodiment, the target RNA is a gene that is involved in HIV. IN another embodiment, the target RNA gene is a gene that is involved in cancer development or progression. In another embodiment, target RNA expression is reduced in a virus infected cell. In another embodiment, the target RNA encodes apoptosis inhibitor proteins and the cells are infected with HIV. RNAi is a method for decreasing the cellular expression of specific proteins of interest (reviewed in Tuschl, ChemBioChem 2:239-245, 2001; Sharp, Gene Dev 15:485-490, 2000; Hutvagner and Zamore, Curr Opin Genet Devel 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the disclosure, a double-stranded RNA (dsRNA) molecule is made that includes between eight and nineteen consecutive nucleobases of a nucleobase oligomer of the disclosure. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Gene Dev 16:948-958, 2002. Paul et al. Nat Biotechnol 20:505-508, 2002; Sui et al. Proc Natl Acad Sci USA 99:5515-5520, 2002; Yu et al. Proc Natl Acad Sci USA 99:6047-6052, 2002; Miyagishi et al. Nat Biotechnol 20:497-500, 2002; and Lee et al. Nat Biotechnol 20:500-505, 2002, each of which is hereby incorporated by reference. In certain embodiments, the sense strand of the double stranded siRNA is split into two smaller oligonucleotides, also referred to as three stranded siRNA.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

The invention encompasses stabilized R/DNA NPs having modifications that protect against 3′ and 5′ exonucleases as well as endonucleases. Such modifications desirably maintain target affinity while increasing stability in vivo. In various embodiments, R/DNA NPs of the invention include chemical substitutions at the ribose and/or phosphate and/or base positions of a given nucleobase sequence. For example, R/DNA NPs of the invention include chemical modifications at the 2′ position of the ribose moiety, circularization of the aptamer, 3′ capping and ‘spiegelmer’ technology. R/DNA NPs having A and G nucleotides sequentially replaced with their 2′-OCH3 modified counterparts are particularly useful in the methods of the invention. Such modifications are typically well tolerated in terms of retaining affinity and specificity. In various embodiments, R/DNA NPs include at least 10%, 25%, 50%, or 75% modified nucleotides. In other embodiments, as many as 80-90% of the R/DNA NPs' nucleotides contain stabilizing substitutions. In other embodiments, 2′-OMe containing R/DNA NPs are synthesized. Such R/DNA NPs are desirable because they are inexpensive to synthesize and natural polymerases do not accept 2′-OMe nucleotide triphosphates as substrates so that 2′-OMe nucleotides cannot be recycled into host DNA. Using methods described herein, R/DNA NPs will be selected for increased in vivo stability. In one embodiment, R/DNA NPs having 2′-F and 2′-OCH3 modifications are used to generate nuclease resistant aptamers. In other embodiments, the nucleic acids of the invention have one or more locked nucleic acids (LNA). LNA refers to a modified RNA nucleotide. The ribose of the LNA is modified with an extra bridge connecting the 2′ oxygen and the 4′ carbon which locks the ribose into the North or 3′-endo conformation. See e.g., Kaur, H. et al., Biochemistry, vol. 45, pages 7347-55; and Koshkin, A. A., et al., Tetrahedron, vol. 54, pages 3607-3630. In other embodiments, one or more nucleic acids of the invention incorporate a morpolino structure where the nucleic acid bases are bound to morpholine rings instead of deoxyribose rings and are linked through phosphorodiamidate groups instead of phosphates. See e.g., Summerton, J. and Weller, D., Antisense & Nucleic Acid Drug Development, vol. 7, pages 187-195. Yet other modifications, include (PS)-phosphate sulfur modifications wherein the phosphate backbone of the nucleic acid is modified by the substitution of one or more sulfur groups for oxygen groups in the phosphate backbone. Other modifications that stabilize nucleic acids are known in the art and are described, for example, in U.S. Pat. No. 5,580,737; and in U.S. Patent Application Publication Nos. 20050037394, 20040253679, 20040197804, and 20040180360.

The agent may be a RNA or DNA aptamer. An aptamer is a stable DNA, RNA, or peptide that binds with high affinity and specificity to targets such as small organics, peptides, proteins, cells, and tissues. Unlike antibodies, some aptamers exhibit stereoselectivity. The present invention is not limited to any particular aptamer, but rather can be any aptamer known in the art to be useful in treating a disease or condition. For example, the Aptamer Database is a comprehensive, annotated repository for information about aptamers and in vitro selection. This resource is provided to collect, organize and distribute all the known information regarding aptamer selection, and is publicly available at http://aptamer.icmb.utexas.edu/.

The agent may be RNA-DNA hybrids with split functionalities, as described infra.

The agent may also be a targeting agent that directs the nanoparticle to a delivery site. For example, the targeting agent may be a ligand, e.g. a peptide ligand that has specific cell surface binding partners, e.g., ligand receptors, that are preferentially exhibited on the surface of a target cell. As used herein, “receptor” and “ligand” refer to two members of a specific binding pair that are binding partners. A receptor is that member of the pair that is found localized on the surface of the target; the ligand is the member of the pair that is found on the surface of the nanoparticle. Accordingly, the in certain embodiments, the invention features a nanoparticle comprising a member of a binding pair, or a fragment thereof that retains the capacity to specifically bind the other member of the binding pair, on its surface and the other member of that binding pair, or a fragment thereof that retains the capacity to specifically bind its partner, is present on the surface of a target. In certain embodiments, the targeting agent may be an antibody, for example a single-chain antibody, for which a binding partner would include an antigen thereof, or a fragment, derivative or variant thereof that retains the capacity to bind to the single-chain antibody.

A therapeutic agent may be a molecule, atom, ion, receptor and/or other entity which is capable of detecting, identifying, inhibiting, treating, catalyzing, controlling, killing, enhancing or modifying a target such as a protein, glyco protein, lipoprotein, lipid, a targeted cell, a targeted organ, or a targeted tissue.

In certain cases, the therapeutic agent is a radiotherapeutic agent, and can be selected from, but is not limited to radioactive gadolinium, radioactive boron, and radioactive iodine.

In certain examples, the agent can be, but is not limited to: drugs, such as antibiotics, analgesics, hypertensives, cardiotonics, and the like, such as acetaminaphen, acyclovir, alkeran, amikacin, ampicillin, aspirin, bisantrene, bleomycin, neocardiostatin, carboplatin, chloroambucil, chloramphenicol, cytarabine, daunomycin, doxorubicin, fluorouracil, gentamycin, ibuprofen, kanamycin, meprobamate, methotrexate, novantrone, nystatin, oncovin, phenobarbital, polymyxin, probucol, procarbabizine, rifampin, streptomycin, spectinomycin, symmetrel, thioguanine, tobramycin, temozolamide, trimethoprim, cisplatin, oxaliplatin, mechlorethamine, cyclophosphamide, chlorambucil, azathioprine, mercaptopurine, vinca alkaloids, taxanes, vincristine, vinblastine vinorelbine, vindesine, etoposide, teniposide, paclitaxel, irinotecan, topotecan, amsacrine, etoposide, etoposide phosphate, teniposide, and dactinomycinand valban; diphtheria toxin, gelonin, exotoxin A, abrin, modeccin, ricin, radioactive gadolinium, radioactive boron, and radioactive iodine; or toxic fragments thereof; metal ions, such as the alkali and alkaline-earth metals; radionuclides, such as those generated from actinides or lanthanides or other similar transition elements or from other elements, such as 51Cr, 47 Sc, 67 Cu, 67 Ga, 82 Rb, 89 Sr, 88 Y, 90 Y, 99m Tc, 105 Rh, 109 Pd, 111 In, 115m In, 125 I, 131 I, 140 Ba, 140 La, 149 Pm, 153 Sm, 159 Gd, 166 Ho, 175 Yb, 177 Lu, 186 Re, 188 Re, 194 Ir, and 199 Au; signal generators, which includes anything that results in a detectable and measurable perturbation of the system due to its presence, such as fluorescing entities, phosphorescence entities and radiation; signal reflectors, such as paramagnetic entities, for example, Fe, Gd, Cr, or Mn; chelated metal, such as any of the metals given above, whether or not they are radioactive, when associated with a chelant; signal absorbers, such as contrast agents and electron beam opacifiers, for example, Fe, Gd, Cr, or Mn; antibodies, including monoclonal antibodies and anti-idiotype antibodies; antibody fragments; hormones; biological response modifiers such as interleukins, interferons, viruses and viral fragments; diagnostic opacifiers; and fluorescent moieties. Other pharmaceutical materials include scavenging agents such as chelants, antigens, antibodies or any moieties capable of selectively scavenging therapeutic or diagnostic agents.

Other examples of therapeutic agents include antimicrobial agents, analgesics, antiinflammatory agents, counterirritants, coagulation modifying agents, diuretics, sympathomimetics, anorexics, antacids and other gastrointestinal agents; antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or a combination thereof. Other suitable therapeutic moieties include contraceptives and vitamins as well as micro- and macronutrients. Still other examples include antiinfectives such as antibiotics and antiviral agents; analgesics and analgesic combinations; anorexics; antiheimintics; antiarthritics; antiasthmatic agents; anticonvulsants; antidepressants; antidiuretic agents; antidiarrleals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Nanoparticles may be directed to target sites. Preferred target sites comprise cancer cells, solid tumors, sites of inflammation and damaged bone or tissue.

For example, nanoparticle may further comprise an antibody or a peptide that acts as a targeting moiety to enable specific binding to a target cell bearing a target molecule, e.g., a cell surface marker to which the antibody or peptide is directed or a disease-specific marker to which the antibody or peptide is directed. The nanoparticle may further comprise a nucleotide, e.g. an oligonucleotide that acts as a targeting moiety to enable specific binding to a target cell bearing a target molecule. For example, the oligonucleotide may be an aptamer that binds a specific target molecule.

Further exemplary potential applications of the multi-functional nanoparticles of the invention include use of the nanoparticles as riboswitch aptamers, ribozymes, or beacons.

Riboswitches are a type of control element that use untranslated sequence in an mRNA to form a binding pocket for a metabolite that regulates expression of that gene. Riboswitches are dual function molecules that undergo conformational changes and that communicate metabolite binding typically as either increased transcription termination or reduced translation efficiency via an expression platform.

Ribozymes catalyze fundamental biological processes, such as RNA cleavage by transesterification. The polyvalent RNA nanoparticles of the invention can be incorporated in to ribozymes using methods described in, for example, U.S. Pat. No. 6,916,653, incorporated by reference in its entirety herein.

A number of “molecular beacons” (often fluorescence compounds) can be attached to RNA nanoparticles of the invention to provide a means for signaling the presence of, and quantifying, a target analyte. Molecular beacons, for example, employ fluorescence resonance energy transfer-based methods to provide fluorescence signals in the presence of a particular analyte/biomarker of interest. In preferred embodiments, the term “molecular beacon” refers to a molecule or group of molecules (i.e., a nucleic acid molecule hybridized to an energy transfer complex or chromophore(s)) that can become detectable and can be attached to a nanoparticle under preselected conditions. Similarly, amplifying fluorescent polymers (AFPs) can be utilized in the present invention. An AFP is a polymer containing several chromophores that are linked together. As opposed to isolated chromophores that require 1:1 interaction with an analyte in conventional fluorescence detection, the fluorescence of many chromophores in an AFP can be influenced by a single molecule. For example, a single binding event to an AFP can quench the fluorescence of many polymer repeat units, resulting in an amplification of the quenching. Quenching is a process which decreases the intensity of the fluorescence emission. Molecular beacons and AFPs, including their methods for preparation, that can be used in the present invention are described in numerous patents and publications, including U.S. Pat. No. 6,261,783.

Any protein can be coupled to nanoparticles. For instance, glycoproteins are most easily coupled, as they can be oxidized to generate an active aldehyde group. Other proteins can be coupled via their —COOH group(s) but with lower efficiency. However, other means known in the art, such as di-imide reagents, e.g. carbodiimide can be used to couple proteins lacking sugars to the nanoparticles.

Polyethylene Glyocol (PEG) chains can be conjugated to the nanoparticles. PEG chains render the nanotubes highly water-soluble. PEG-phospholipids (PEG-PL) have been used in the formation of micelles and liposomes for drug delivery (Adlakha-Hutcheon, G.; Bally, M. B.; Shew, C. R.; Madden, T. D. Nature Biotech. 1999, 17, 775-779; Meyer, 0.; Kirpotin, D.; Hong, K.; Sternberg, B.; Park, J. W.; Woodle, M. C.; Papahadjopoulos, D. J. Biol. Chem. 1998, 273, 15621-15627; Papahadjopoulos, D.; Allen, T. M.; Gabizon, A.; Mayhew, E.; Matthay, K.; Huang, S. K.; Lee, K. D.; Woodle, M. C.; Lasic, D. D.; Redemann, C.; Martin, F. J. Proc. Nat. Acad. Sci. USA. 1991, 88, 11460-11464).

Functional groups can be coupled to the nanoparticle, for instance the functional group can be a reactive functional group. Suitable functional groups include, but are not limited to, a haloacetyl group, an amine, a thiol, a phosphate, a carboxylate, a hydrazine, a hydrazide an aldehyde or a combination thereof. Other functional groups include groups such as a reactive functionality or a complementary group. In addition, RNA functional groups can be attached, as for example ribozymes or riboswitch aptamers.

The nanoparticle can be used for attachment of small molecules for specific interactions with nucleic acids, carbohydrates, lipids, proteins, antibodies, or other ligands.

The nanoparticle can have dyes attached. The dye is can be a fluorescent dye, or a plurality of fluorescent dyes. Suitable dyes include, but are not limited to, YOYO-1, JOJO-1, LOLO-1, YOYO-3, TOTO, BOBO-3, SYBR, SYTO, SYTOX, PicoGreen, OliGreen, and combinations thereof. Other dyes include, thiazole orange, oxazole yellow, or non-intercalating dyes such as fluorescein, rhodamine, cyanine or coumarin based dyes, and combinations thereof. Other suitable dyes include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,-2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy 5; Cy 5.5; Cy 7; IRD 700; IRD 800; La Jolla Blue; phthalocyanine; and naphthalo cyanine. Suitable dyes for use in the nanoparticles of the present invention include, without limitation, a family of homodimeric cyanine DNA intercalating dyes from Molecular Probes that cover the visible spectrum, such as YOYO-1 (488/509), JOJO-1 (532/545), LOLO-1 (565/579), and YOYO-3 (612/631), SYBR-101 (488/505) and SYTO-62 (652/676). Given sufficient detection SN, dyes are mixed in various ratios in a single particle such that, for example, different fluorescence spectra are obtained from mixtures of just 2 dyes. According to the invention, one or more therapeutic, diagnostic, or delivery agents are directly included in the building block sequences. In certain embodiments, the delivery agent can be a targeting agent. Targeting agents are used to direct the nanoparticle to a tissue or cell target. An exemplary embodiment of a targeting agent is an antibody. For example, antibodies suitable for use as targeting agents in the present invention include antibodies directed to cell surface antigens which cause the antibody-nanoparticle complex to be internalized, either directly or indirectly. For example, in the treatment of cancer, suitable antibodies include antibodies to CD33 and CD22. CD33 and CD22 that are over-expressed and dimerized on lymphomas.

In certain preferred embodiments of the invention biotin is conjugated to the nanoparticle. For example, the nanoparticles of the invention can be further functionalized using biotin-streptavidin interactions to immobilize molecules inside or outside the polyhedra, e.g. polyhedral cages. For example, streptavidin can be conjugated to guanosine mono-phosphothioate (GMPS)-modified tectoRNAs by means of a biotin linker. In certain preferred embodiments, the biotin linker is incorporated to a mono-phosphothioate at the 5′ position of tectoRNAs.

A wide variety of particle sizes are suitable for the present invention. In certain aspects, the particle has a diameter of about 10 nanometers to about 10 microns. Preferably the particle diameter is about 10 to 700 nanometers, and more preferably, the diameter of about 10 nanometers to about 100 nanometers.

The polyvalent RNA nanoparticle or the polyvalent RNA nanotube as described herein has a number of uses. For example, the polyvalent RNA nanoparticle or the polyvalent RNA nanotube can be used in drug delivery, imaging, nanocircuits, cell growth surfaces, medical implants, medical testing, or gene therapy.

In one particular embodiment, the polyvalent RNA nanoparticle or the polyvalent RNA polyhedra, e.g., cages, as described can be used in biological meshes. In one exemplary embodiment, the invention as described herein may find use as a biosensor in, for example, pathogen detection. In one particular embodiment, self-assembling nano-meshes are used to attach biosensors for pathogen detection or for x-ray crystallography by placing multiple copies of a protein or functional RNAs, for example, on the mesh. Biosensors for pathogen detection are advantageously employed in bioterrorism capacities.

In another exemplary embodiment, the polyvalent nanoparticles of the invention, as described herein, are employed as skeletons or scaffolds for tissue growth. These uses are exemplary, and not considered to be limiting.

### Compositions

The invention, in part, pertains to a drug delivery composition comprising the nanoparticles or the activatable nanoparticle systems as described herein. The drug delivery composition of the invention can gain entry into a cell or tissue.

Advantageously, the drug delivery composition of the invention provides for a more controlled delivery of an active agent, especially a therapeutic agent, to a site of action at an optimum rate and therapeutic dose. Thus, improvements in therapeutic index may be obtained by modulating the distribution of the active ingredient in the body. Association of the active ingredient with a delivery system enables, in particular, its specific delivery to the site of action or its controlled release after targeting the action site. By reducing the amount of active ingredient in the compartments in which its presence is not desired, it is possible to increase the efficacy of the active ingredient, to reduce its toxic side effects and even modify or restore its activity.

It is understood by one of skill in the art that changing the base composition of RNA changes the half-life of RNA and thus the release of RNA from the composition. For instance, the composition can be modified to consist of fast release, slow release or a staged release of polyvalent RNA nanoparticle.

In certain preferred embodiments, the drug delivery composition can comprise a second therapeutic agent. In some embodiments, the composition comprising nanoparticles and the second therapeutic agent are administered simultaneously, either in the same composition or in separate compositions. In some embodiments, the nanoparticle composition and the second therapeutic agent are administered sequentially, i.e., the nanoparticle composition is administered either prior to or after the administration of the second therapeutic agent. The term “sequential administration” as used herein means that the drug in the nanoparticle composition and the second agent are administered with a time separation of more than about 15 minutes, such as more than about any of 20, 30, 40, 50, 60 or more minutes. Either the nanoparticle composition or the chemotherapeutic agent may be administered first. The nanoparticle composition and the chemotherapeutic agent are contained in separate compositions, which may be contained in the same or different packages. In some embodiments, the administration of the nanoparticle composition and the second therapeutic agent are concurrent, i.e., the administration period of the nanoparticle composition and that of the second therapeutic agent overlap with each other. In some embodiments, the administration of the nanoparticle composition and the second therapeutic agent are non-concurrent. For example, in some embodiments, the administration of the nanoparticle composition is terminated before the second therapeutic agent is administered. In some embodiments, the administration of the second therapeutic agent is terminated before the nanoparticle composition is administered. Administration may also be controlled by designing the RNA nanoparticle or nano-tube to have different half-lives. Thus, particle dissolution would be controlled by a timed release based upon variations in designed RNA stability.

The second therapeutic agent is selected from, but not limited to chemotherapeutic agents, cardiovascular drugs, respiratory drugs, sympathomimetic drugs, cholinomimetic drugs, adrenergic or adrenergic neuron blocking drugs, analgesics/antipyretics, anesthetics, antiasthmatics, antibiotics, antidepressants, antidiabetics, antifungals, antihypertensives, anti-inflammatories, antianxiety agents, immunosuppressive agents, immunomodulatory agents, antimigraine agents, sedatives/hypnotics, antianginal agents, antipsychotics, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterials, antivirals, antimicrobials, anti-infectives, bronchodialators, hormones, hypoglycemic agents, hypolipidemic agents, proteins, peptides, nucleic acids, agents useful for erythropoiesis stimulation, antiulcer/antireflux agents, antinauseants/antiemetics and oil-soluble vitamins, or combinations thereof.

When the second therapeutic agent is a chemotherapeutic agent, the chemotherapeutic agent is selected from, but not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlomaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfomithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

Reference to a chemotherapeutic agent herein applies to the chemotherapeutic agent or its derivatives and accordingly the invention contemplates and includes either of these embodiments (agent; agent or derivative(s)). “Derivatives” or “analogs” of a chemotherapeutic agent or other chemical moiety include, but are not limited to, compounds that are structurally similar to the chemotherapeutic agent or moiety or are in the same general chemical class as the chemotherapeutic agent or moiety. In some embodiments, the derivative or analog of the chemotherapeutic agent or moiety retains similar chemical and/or physical property (including, for example, functionality) of the chemotherapeutic agent or moiety.

The invention also relates to pharmaceutical or diagnostic compositions comprising the nanoparticles of the invention and a pharmaceutically acceptable carrier. The phrase “pharmaceutically acceptable carrier” is art recognized and includes a pharmaceutically acceptable material, composition or vehicle, suitable for administering compounds used in the methods described herein to subjects, e.g., mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in Remington's Pharmaceutical Sciences, Mack Publishing Company, a standard reference text in this field.

### Methods of Treatment

The invention encompasses methods of treating or preventing diseases or disorders by administering to subjects in need thereof an effective amount of an activatable nanoparticle system described herein or a composition comprising same. Accordingly, a number of diseases or disorders are suitable for treatment according to the methods of the invention. Examples include, but are not limited to, Adenoma, Ageing, AIDS/HIV, Alopecia, Alzheimer's disease, Anemia, Arthritis, Asthma, Atherosclerosis, Cancer, Cardiac conditions or disease, Diabetes mellitus, Foodborne illness, Hemophilia A-E, Herpes, Huntington's disease, Hypertension, Headache, Influenza, Multiple Sclerosis, Myasthenia gravis, Neoplasm, Obesity, Osteoarthritis, Pancreatitis, Parkinson's disease, Pelvic inflammatory disease, Peritonitis, Periodontal disease, Rheumatoid arthritis, Sepsis, Sickle-cell disease, Teratoma, Ulcerative colitis, and Uveitis.

The methods of the invention further encompass diagnostics.

The methods may be practiced in an adjuvant setting. “Adjuvant setting” refers to a clinical setting in which, for example, an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated. The methods provided herein may also be practiced in a neoadjuvant setting, i.e., the method may be carried out before the primary/definitive therapy. Thus, in some embodiments, the individual has previously been treated. In other embodiments, the individual has not previously been treated. In some embodiments, the treatment is a first line therapy.

### Dosage

Human dosage amounts of compositions of the activatable nanoparticle systems described herein can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Methods of Delivery

The activatable nanoparticle compositions described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the nanoparticle composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the nanoparticle composition is administrated intravenously. In some embodiments, the nanoparticle composition is administered orally.

The dosing frequency of the administration of the nanoparticle composition depends on the nature of the therapy and the particular disease being treated. For example, dosing frequency may include, but is not limited to, once daily, twice daily, weekly without break; weekly, three out of four weeks; once every three weeks; once every two weeks; weekly, two out of three weeks.

The administration of nanoparticles may be carried out at a single dose or at a dose repeated once or several times after a certain time interval. The appropriate dosage varies according to various parameters, for example the individual treated or the mode of administration.

The dosing frequency of the nanoparticle composition or the nanoparticle composition and the second therapeutic agent may be adjusted over the course of the treatment, based on the judgment of the administering physician.

When administered separately, the nanoparticle composition and the second therapeutic agent can be administered at different dosing frequency or intervals. For example, the nanoparticle composition can be administered weekly, while a second agent can be administered more or less frequently. In some embodiments, sustained continuous release formulation of the nanoparticle and/or second agent may be used. Various formulations and devices for achieving sustained release are known in the art. The doses required for the nanoparticle composition and/or the second agent may (but not necessarily) be lower than what is normally required when each agent is administered alone. Thus, in some embodiments, a subtherapeutic amount of the drug in the nanoparticle composition and/or the second agent are administered. “Subtherapeutic amount” or “subtherapeutic level” refer to an amount that is less than the therapeutic amount, that is, less than the amount normally used when the drug in the nanoparticle composition and/or the second agent are administered alone. The reduction may be reflected in terms of the amount administered at a given administration and/or the amount administered over a given period of time (reduced frequency).

A combination of the administration configurations described herein can be used. The combination therapy methods described herein may be performed alone or in conjunction with another therapy, such as surgery, radiation, chemotherapy, immunotherapy, gene therapy, and the like. Additionally, a person having a greater risk of developing the disease to be treated may receive treatments to inhibit and/or delay the development of the disease. The dose of nanoparticle composition will vary with the nature of the therapy and the particular disease being treated. The dose should be sufficient to effect a desirable response, such as a therapeutic or prophylactic response against a particular disease. Appropriate doses will be established by persons skilled in the art of pharmaceutical dosing such as physicians.

In certain embodiments, the siRNAs can be administered as bolaamphiphiles. Bolaamphiphiles have relatively low toxicities, long persistence in the blood stream, and most importantly, in aqueous conditions can form poly-cationic micelles thus, becoming amenable to association with siRNAs. Depending on the application, the extent of siRNA chemical protection, delivery efficiency, and further intracellular release can be varied by simply changing the type of bolaamphiphile used (see, e.g. Kim et al. Mol Ther Nucleic Acids. 2: e80, 2013, incorporated by reference in its entirety herein).

### Kits

The disclosure provides kits for the treatment or prevention of disease. In one embodiment, the kit includes a therapeutic or prophylactic composition containing an effective amount of an agent of the invention (e.g., NPs) in unit dosage form. In some embodiments, the kit comprises a sterile container which contains a therapeutic or prophylactic compound; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the disclosure is provided together with instructions for administering it to a subject having or at risk of developing a disease. The instructions will generally include information about the use of the composition for the treatment or prevention of the disease (e.g., neoplasia or viral infection). In other embodiments, the instructions include at least one of the following: description of the compound; dosage schedule and administration for treatment or prevention of the disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Introduction

RNA interference (RNAi) is a cellular process that occurs post-transcriptionally to regulate gene expression. This pathway can be mobilized through the foreign introduction of small interfering RNAs (siRNA) allowing for the regulation of genes that contribute to the diseased state. In order to simultaneously target several genes inside the same diseased cell with multiple siRNAs, those siRNAs need to be co-delivered in a controlled fashion. The field of therapeutic RNA nanotechnology aims to develop novel technologies facilitating this task through the introduction of programmable RNA-based nanoparticles amenable to functionalization with various therapeutics.

These nanoparticles demonstrate a precise and reproducible formulation, modularity, and programmability. However, for further biomedical applications, additional control over their specific intracellular activation is desired in order to minimize any unwanted interactions in vivo. The present invention recently introduced an approach that relies on the use of RNA-DNA hybrids for delivery of multiple split functionalities and their simultaneous intracellular activation (see Afonin, K. A. et al., Activation of different split functionalities on re-association of RNA-DNA hybrids. Nature Nanotechnology, 2013, 8, 296-304).

The methodology involved splitting the functional RNA entities (e.g., RNAi activators) into two non-functional RNA-DNA hybrids. Each DNA strand was designed to be slightly (˜12-nt) longer than its RNA counterpart. Thus, upon the formation of the RNA-DNA hybrids, these duplexes would have ssDNA toeholds that were complementary to each other. The presence of ssDNA toeholds on the extremities of the hybrids allowed for their mutual recognition, re-association, and release of the functional RNA when they were in close proximity. This approach allowed an additional handle on the specificity of the site of action and conferred additional stability in vivo. See Id.

The inventors further demonstrated computationally (see Afonin, K. A., Computational and Experimental Studies of Reassociating RNA/DNA Hybrids Containing Split Functionalities. Methods in Enzymology, 2015, 553, 313-334) and experimentally (Afonin, K. A. et al., Co-transcriptional production of RNA-DNA hybrids for simultaneous release of multiple split functionalities. Nucleic acids research 2014, 42, 2085-2097) that multiple split RNA functionalities could be embedded in the same RNA-DNA hybrids, resulting in their simultaneous co-activation. This was achieved through the introduction, in a controlled fashion, of multiple functional entities, such as RNAi inducers (e.g., up-to seven at once), FRET fluorescence pairs for tracking of re-association in real time, and RNA aptamers for fluorescence response on re-association or specific cell targeting, just to name a few (Rogers, T. A. et al., Fluorescent monitoring of RNA assembly and processing using the split-spinach aptamer. ACS Synthetic Biology, 2015, 4, 162-166).

However, the number of functionalities activated at once was limited by the total length of the DNAs. In addition, the longer DNA duplexes that resulted from re-association became immunogenic (Afonin, K. A. et al., Co-transcriptional production of RNA-DNA hybrids for simultaneous release of multiple split functionalities. Nucleic acids research 2014, 42, 2085-2097). To overcome these limitations, individual hybrids were introduced as the extremities of the compact RNA nanoparticles, nanorings and nanocubes, which were previously designed and characterized as programmable RNA nanoscaffolds. (See Afonin, K. A. et al., Design and self-assembly of siRNA-functionalized RNA nanoparticles for use in automated nanomedicine. Nature Protocols, 2011, 6, 2022-2034. Also see Afonin, K. A. et al., In vitro assembly of cubic RNA-based scaffolds designed in silico, Nature Nanotechnology, 2010, 5, 676-682. Also see Afonin, K. A. et al., Computational and experimental characterization of RNA cubic nanoscaffolds. Methods 2014, 67, 256-265. Also see Grabow, W. W et al. Self-assembling RNA nanorings based on RNAI/II inverse kissing complexes. Nano Letters 2011, 11, 878-887).

For all of the approaches described above, the ssDNA toeholds had to be specifically designed to avoid any internal secondary structures and any undesired interactions with the scaffold strands or split functions while triggering the re-association of the hybrids at physiological temperature (e.g., 37° C.). These limitations would potentially require computer-aided sequence optimization for each new generation of hybrid nanoparticles.

By contrast, as described herein, the methodology disclosed in this invention schematically depicted in FIG. TA relies solely on the use of RNA-DNA hybrids with ssRNA toeholds and demonstrates a significant simplification of all design principles in the existing technique. The overall size of the new nanoparticles amenable to intracellular activation becomes significantly smaller (e.g., up to 120 fewer nucleotides per nanoparticle) compared to designs relying on ssDNA toeholds. In addition, the current method using ssRNA toeholds allows better yields in the co-transcriptional assemblies of the hybrid nanoparticles. Also, a novel computation algorithm aiming at the prediction of multi-stranded hybrid secondary structures and their further re-association was developed to confirm the experimental results.

### Example 1: Rational Design of RNA-DNA Hybrids

As the proof of concept, the function of a Dicer Substrate RNA (DS RNA) was split and was designed to downregulate the production of green fluorescent protein (GFP) (Rose et al. Nucleic acids research 2005, 33, 4140-4156) that is stably expressed in model human breast cancer cells (MDA-MB231/GFP). The use of DS RNAs (as opposed to siRNAs) is required to ensure that once inside the cells, the individual hybrids will not be active in the RNAi pathway (Afonin et al. Nucleic acids research 2014, 42, 2085, 2097). GFP DS RNAs were split between two RNA-DNA hybrids with the DNA strands being 8-, 6-, 4-, and 2-nts shorter than their corresponding complementary RNAs, thus, providing the ssRNA toeholds for further re-association. The hybrids containing the sense strand of DS RNA are referred as H_sen and the hybrids containing the antisense strand are referred as H_ant. A scheme explaining the re-association of new hybrids studied in this work is shown in FIG. 1A.

### Example 2: Re-Association of RNA-DNA Hybrids

Four sets of cognate RNA-DNA hybrids with different ssRNA toehold lengths (2-, 4-, 6-, and 8-nt) were prepared and tested in parallel (FIGS. 1A-1C). The re-association of the hybrids was first assessed by native-PAGE experiments (FIG. 1C). The results show that the extent of re-association is dependent on the length of the ssRNA toeholds. In particular, only partial re-associations were observed for the hybrids with toeholds of 4-nt and less. In silico predictions based on a novel multi-strand secondary structure prediction approach confirmed these results.

To trace the re-association of hybrids in solution in real time, Forster resonance energy transfer (FRET) was measured. The kinetics of re-association were studied using fluorescently labeled (with Alexa 488 and Alexa 546) RNAs entering the composition of the different hybrids. When two fluorescently labeled hybrids are mixed and incubated at 37° C., their re-association brings Alexa 488 within the Forster radius (R₀=6.31 nm) of Alexa 546 (FIG. 1C). Consequently, when excited at 460 nm, the emission of Alexa 488 significantly drops while the fluorescent signal of Alexa 546 increases (FIG. 1C). The results indicate strong dependence of the extent of re-association on the toehold lengths and confirm the previous observation.

### Example 3: Re-Association of RNA-DNA Hybrids in Human Cells

The ability of the hybrids with ssRNA toeholds to enter and re-associate inside mammalian cells was assessed. Fluorescently labeled hybrids were co-transfected into human breast cancer cells and analyzed with confocal microscopy the next day (FIG. 1D) (Afonin et al ACS nano 2015, 9, 251-259; Afonin et al Nano letters 2014, 14, 5662-5671; Afonin et al Nature nanotechnology 2013, 8, 296-304). The samples were excited at 488 nm and the emission of Alexa546 was collected. To estimate the extent of intracellular FRET, Alexa546 sensitized emission was imaged as detailed in our previous work (Afonin et al Nature nanotechnology 2013, 8, 296-304). The FRET signal remaining upon bleed-through correction was calculated and is shown in blue (FIG. 1D, images 1+4 and 5). The ssRNA-toehold driven intracellular re-association of RNA-DNA hybrids was further confirmed by specific gene silencing experiments with human breast cancer cells stably expressing GFP. First, cells were co-transfected with only one hybrid at a time (H_ant), and three days later, the level of eGFP expression was analyzed with fluorescence microscopy and flow cytometry. All experiments were repeated in triplicates, and results demonstrated no GFP silencing caused by the individual hybrids. However, when the same cells were co-transfected with separately prepared complexes of Lipofectamine 2000 (L2K) and individual hybrids (H_sen/L2K and H_ant/L2K), significant GFP silencing was observed (FIG. 1E). It was hypothesized that the longer ssRNA toeholds would be positively correlated with a higher level of re-association and gene silencing. Interestingly, the extent of silencing was proportional to the ssRNA toehold lengths only at relatively low concentrations (1 nM) but not at the higher ones (30 nM). Even though the re-association was not detected for hybrids with 2-nts toeholds at 1 μM concentrations (FIG. 1B-1C), significant gene silencing was observed for the same hybrids at 30 nM concentrations. This discrepancy can be explained by the use of the polycationic carrier (L2K) that electrostatically attracts high quantities of individual hybrids during the transfection and incubation step, and then releases them intracellularly in much smaller volumes (in endosomes), thus, providing a significant increase in the “local” concentration of cognate hybrid. To test the feasibility of this existing hypothesis, several re-association experiments at higher hybrid concentrations were carried; these showed that at high enough concentrations (5 μM or higher), the re-association occurs even between the hybrids with 2-nts ssRNA toeholds.

To demonstrate the generality and the therapeutic potential of this approach, we tested two additional sets of hybrids designed against the full-length genomic HIV-1 RNA that also serves as the mRNA coding for the viral structural proteins and enzymes (Berkhout et al Antiviral research 2011, 92, 7-14.; Liu et al Molecular therapy: the journal of the American Society of Gene Therapy 2009, 17, 1712-1723; Low et al Molecular therapy: the journal of the American Society of Gene Therapy 2012, 20, 820-828). Gag and LDR hybrids targeted the sequences coding the capsid domain and the amino terminus of the matrix domain of Gag respectively (FIG. 2A-2B). Both hybrids were designed to have 8-nt ssRNA toeholds. Transfection of the individual hybrids showed no significant effect on relative virus release. However, the co-transfection of cognate hybrids reduced the production of virus by 65% to 75% (FIG. 2C). The total amount of Gag was reduced on average by 65% with the reassociated hybrids, while the cellular expression levels of glyceraldehyde 3-phosphate dehydrogenase (GAPDH) were not affected.

### Example 4: Controlled Activation of RNAi and by Nanorings Functionalized with RNA-DNA Hybrids

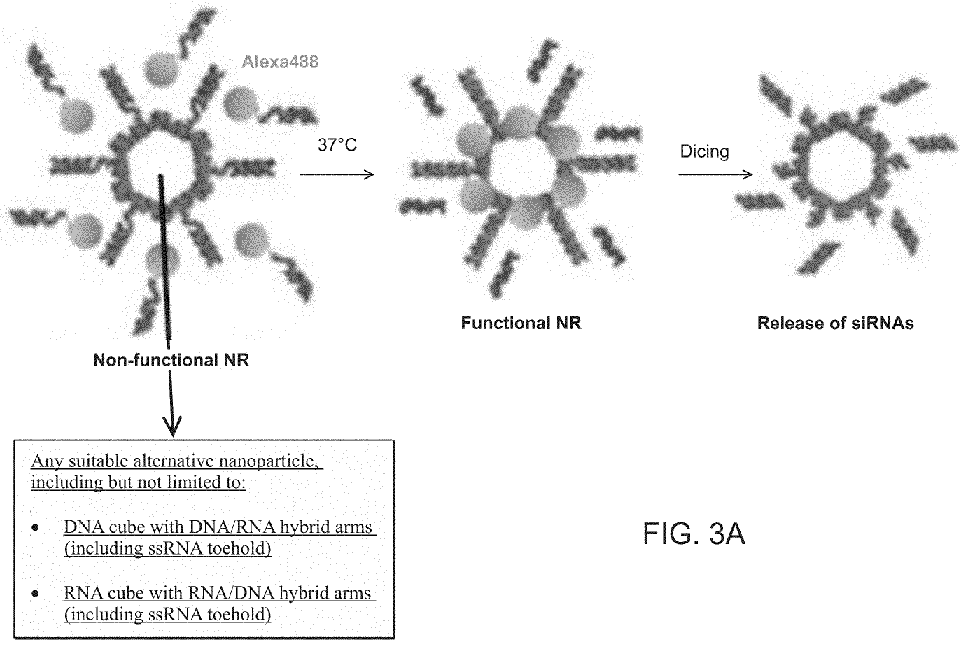

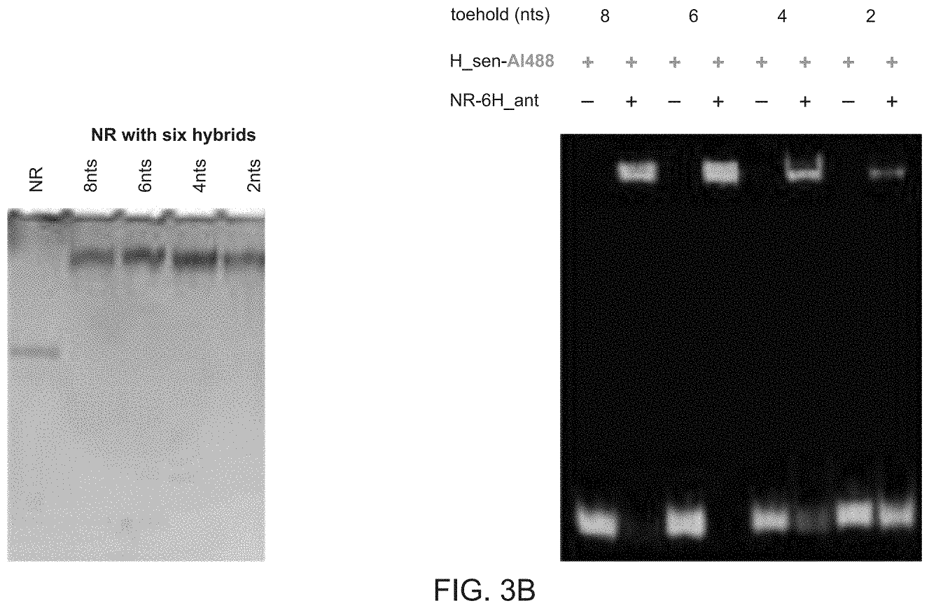

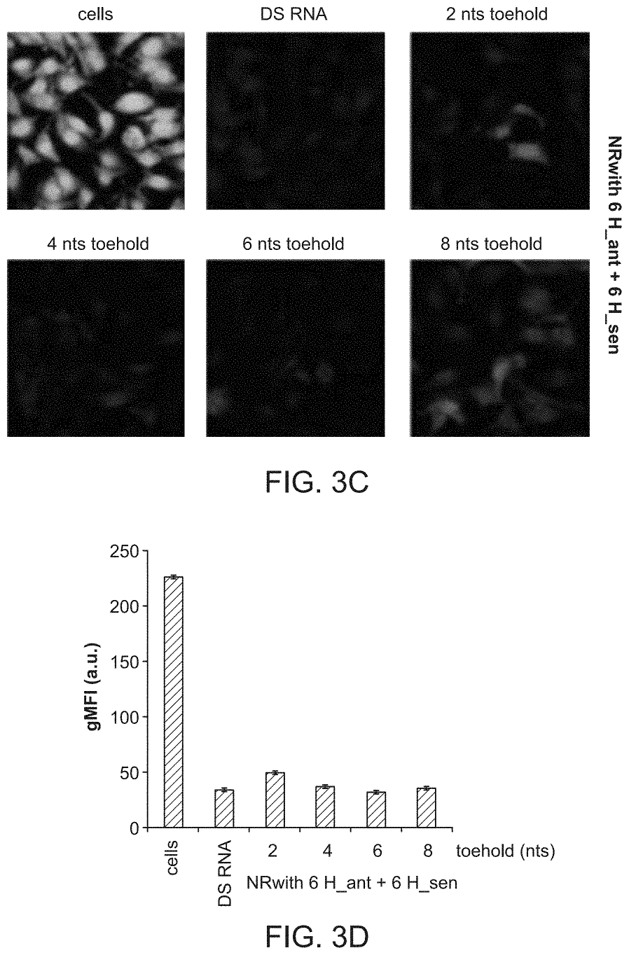

To guarantee the simultaneous delivery and intracellular activation of multiple RNA-DNA hybrids, the previously characterized nanorings were employed (Grabow et al Nano letters 2011, 11, 878-887). Each nanoring was functionalized with six RNA-DNA hybrids of varying ssRNA toehold lengths (2-, 4-, 6-, and 8-nts in length). The toeholds were oriented towards the nanoring scaffolds to improve the stability of ssRNAs (exonuclease degradation). All nanoparticles were produced co-transcriptionally (Afonin et al Nature nanotechnology 2010, 5, 676-682; Afonin et al Nano letters 2012, 12, 5192-5195; Afonin et al Nucleic acids research 2012, 40, 2168-2180; Geary et al Science 2014, 345, 799-804), gel purified, recovered and further characterized. The relative yields of the co-transcriptionally produced nanoparticles with ssRNA toeholds were higher than the yields of the previously characterized nanoparticles (Afonin et al Nano letters 2014, 14, 5662-5671) containing ssDNA toeholds. All assemblies were confirmed by native-PAGE and their re-association was visualized using fluorescently labeled cognate hybrids (FIG. 3B). In agreement with previous results, the assemblies with 4- and 2-nts ssRNA toeholds demonstrated only limited re-association and the 6- and 8-nts toeholds were the most effective in forming the fully functionalized nanorings (with six DS RNAs). The formation of functional rings was further supported by cell transfection experiments (FIG. 3C).

### Example 5: Computational Studies of RNA-DNA Hybrid Re-Association

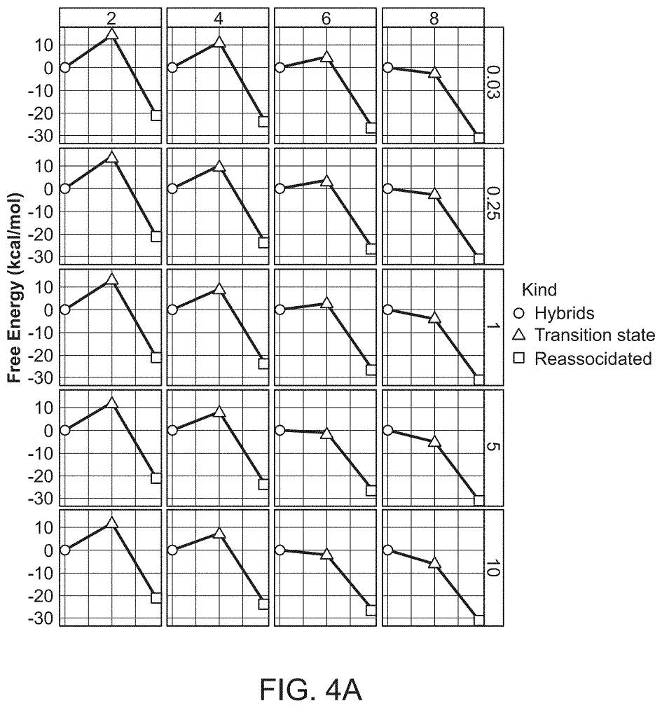

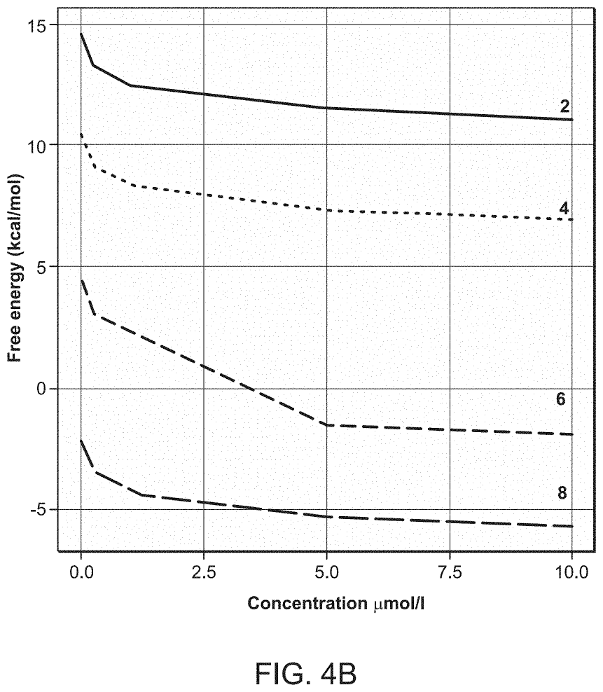

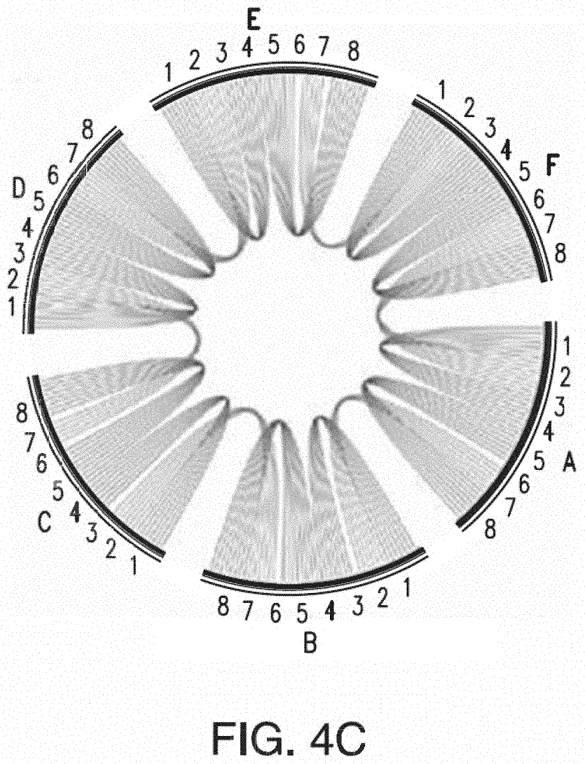

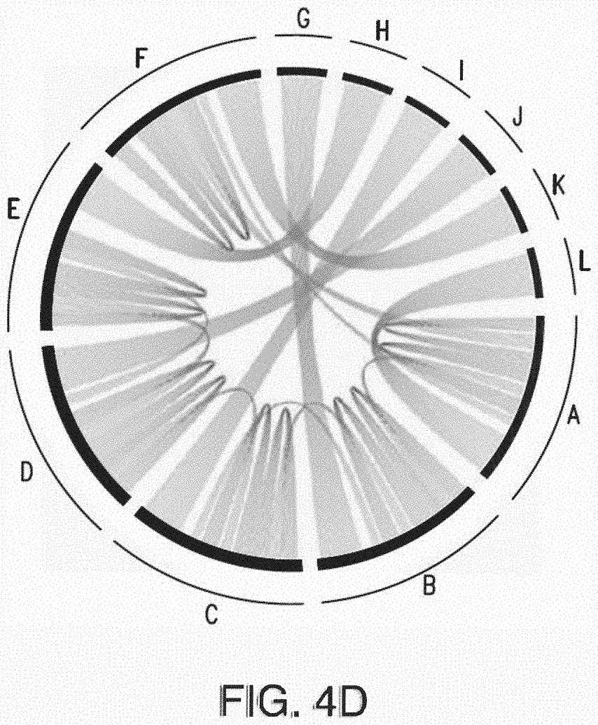

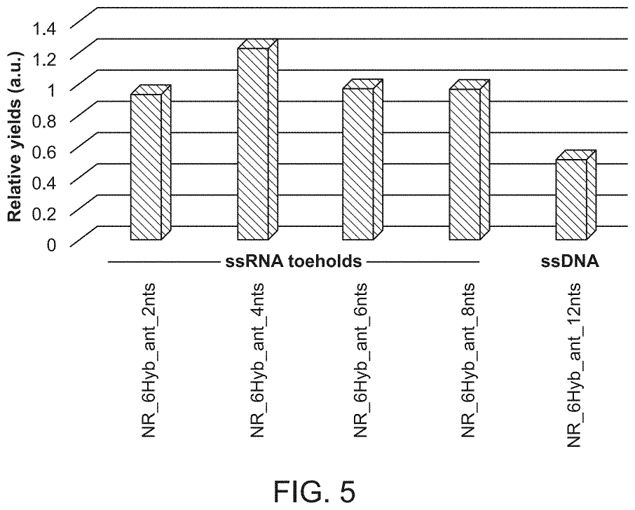

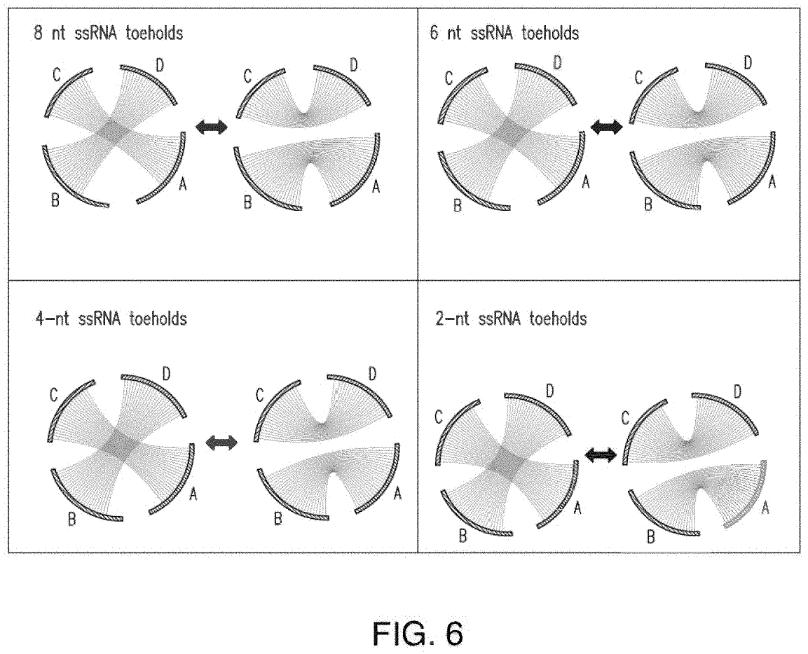

The estimated free energies of the non-re-associated hybrid state, the re-associated state and a quadruplex intermediate state are shown in FIG. 4A for different toehold lengths and for different concentrations. Interestingly, the computational thermodynamic predictions indicate that all toehold strand lengths should lead to re-association, because the re-associated state has a lower free energy compared to the RNA/DNA hybrids (FIG. 4A). Estimating, however, the free energy of activation of the transition state (a model of a quadruplex conformation consisting of non-re-associated RNA/DNA hybrid helices but bound cognate toeholds was used), suggests that hybrid sequences with toehold lengths of less than 6 nt are prevented from re-association because of an energetic barrier, possibly leading to kinetic frustration. Conversely, there is for high concentrations and toehold lengths greater 4 nt (according to the computational model), no kinetic barrier with respect to the model transition state (FIG. 4A-4B).

Furthermore, the computational structure predictions for the RNA nanoring scaffold sequences (FIG. 4C) correspond to a ring structure connected by kissing loop interactions. A secondary structure prediction of the functionalized nanoring sequences with cognate DNAs (FIG. 4D) shows the expected strand interactions with the exception of one kissing loop interaction.

The promise of RNA interference based therapeutics is made evident by the recent surge of biotechnological drug companies that pursue such therapies and their progression into human clinical trials. Recent achievements in RNA nanotechnology introduced nanoscaffolds (nanorings) with the potential for a broad use in biomedical applications (PCT/US10/38818, incorporated by reference in its entirety herein). As presented herein, besides functionalization with multiple short interfering RNAs for combinatorial RNA interference, these nanoscaffolds also allow simultaneous embedment of assorted RNA aptamers, fluorescent dyes, proteins, as well as recently developed auto-recognizing RNA-DNA hybrids used to conditionally activate multiple split functionalities. These new constructs were extensively characterized and visualized in vitro, in cell culture and in vivo by various experimental techniques. The results revealed a higher detection sensitivity of diseased cells and significant increases in silencing efficiencies of targeted genes compared to the silencing caused by equal amounts of conventional siRNAs. Due to the combinatorial nature and relative engineering simplicity, these RNA nanoparticles are expected to be useful for various nanotechnological applications.

### Methods

The foregoing experiments were carried out with, but not limited to, the following methods and materials.

RNA nanoring sequence design assemblies and native PAGE. The detailed design and production of RNA strands entering the composition of nanorings functionalized with six siRNAs is comprehensively described elsewhere(Afonin et al Multifunctional RNA nanoparticles. Nano Letters 2014, 14, 5662-5671). The full list of RNA sequences used is available, and is shown below.

### Sequences

Any suitable sequences of RNA and DNA may be obtained, designed, or otherwise provided by any means to prepare, manufacture, or otherwise assemble the nanoparticles of the invention. For example, RNA and DNA sequences used to assemble RNA-DNA hybrids containing split asymmetric 25/27mer Dicer substrate RNA (DS RNA) duplex designed against eGFP (Rose et al Nucleic acids research 2005, 33, 4140-4156). Exemplary sequences include: DS RNA sense: 5′-pACCCUGAAGUUCAUCUGCACCACcg (SEQ ID NO: 1)

DS RNA sense:
(SEQ ID NO: 1)
5′-pACCCUGAAGUUCAUCUGCACCACcg
sense labeled with Aexa-488:
(SEQ IDS NO: 2)
5′-pACCCUGAAGUUCAUCUGCACCACcg-A1488
DNA for sense(8 nts RNA toehold)
(SEQ ID NO: 3)
5′-CAGATGAACTTCAGGGTca
DNA for sense(6 nts RNA toehold)
(SEQ ID NO: 4)
5′-TGCAGATGAACTTCAGGGTca
DNA for sense(4 nts RNA toehold)
(SEQ ID NO: 5)
5′-GGTGCAGATGAACTTCAGGGTca
DNA for sense(2 nts RNA toehold)
(SEQ ID NO: 6)
5′-GTGGTGCAGATGAACTTCAGGGTca
DS RNA antisense:
(SEQ ID NO: 7)
5′-CGGUGGUGCAGAUGAACUUCAGGGUCA
antisense labeled with Alexa546:
(SEQ ID NO: 8)
5′-A1546-CGGUGGUGCAGAUGAACUUCAGGGUCA
DNA for ant(8 nts RNA toehold)
(SEQ ID NO: 9)
5′-TGACCCTGAAGTTCATCTG
DNA for ant(6 nts RNA toehold)
(SEQ ID NO: 10)
5′-TGACCCTGAAGTTCATCTGCA
DNA for ant(4 nts RNA toehold)
(SEQ ID NO: 11)
5′-TGACCCTGAAGTTCATCTGCACC
DNA for ant(2 nts RNA toehold)
(SEQ ID NO: 12)
5′-TGACCCTGAAGTTCATCTGCACCAC

RNA and DNA sequences used to assemble RNA-DNA hybrids containing split asymmetric DSRNA duplex designed against HIV (Berkhout et al Antiviral research 2011, 92, 7-14; Liu et al Molecular therapy: The Journal of the American Society of Gene Therapy 2009, 17, 1712-1723; Low et al Molecular Therapy: the Journal of the American Society of Gene Therapy 2012, 20, 820-828). The names of corresponding DS RNAs are indicated for each DS RNA: Capsid (GAG) and Primer Binding Site—Matrix (LDR). Exemplary sequences include:

Capsid (Gag)
Sense:
(SEQ ID NO: 13)
5′-pGAAGAAAUGAUGACAGCAUUUCAGG
DNA for GAG sense (RNA toehold):
(SEQ ID NO: 14)
5′-GCTGTCATCATTTCTTCTT
Antisense:
(SEQ ID NO: 15)
5′-CCUGAAAUGCUGUCAUCAUUUCUUCUU
DNA for GAG ant (RNA toehold):
(SEQ ID NO: 16)
5′-AAGAAGAAATGATGACAGC
Primer Binding Site-Matrix (LDR)
Sense:
(SEQ ID NO: 17)
5′-pGGAGAGAGAUGGGUGCGAGUUCGUC
DNA for LDR sense (RNA toehold):
(SEQ ID NO: 18)
5′-CGCACCCATCTCTCTCCTT
Antisense:
(SEQ ID NO: 19)
5′-GACGGACUCGCACCCAUCUCUCUCCUU
DNA for LDR ant (RNA toehold):
(SEQ ID NO: 20)
5′-AAGGAGAGAGATGGGTGCG

RNA nanorings 3′-side functionalized with DS RNA antisenses against Green Fluorescent Protein (Afonin et al Nano Letters 2014, 14, 5662-5671).

A:
(SEQ ID NO: 21)
5′-GGGAACCGUCCACUGGUUCCCGCUACGAGAGCCUGCCUCGUAGCUUCG
GUGGUGCAGAUGAACUUCAGGGUCA
B:
(SEQ ID NO: 22)
5′-GGGAACCGCAGGCUGGUUCCCGCUACGAGAGAACGCCUCGUAGCUUCG
GUGGUGCAGAUGAACUUCAGGGUCA
C:
(SEQ ID NO: 23)
5′-GGGAACCGCGUUCUGGUUCCCGCUACGAGACGUCUCCUCGUAGCUUCG
GUGGUGCAGAUGAACUUCAGGGUCA
D:
(SEQ ID NO: 24)
5′-GGGAACCGAGACGUGGUUCCCGCUACGAGUCGUGGUCUCGUAGCUUCG
GUGGUGCAGAUGAACUUCAGGGUCA
E:
(SEQ ID NO: 25)
5′-GGGAACCACCACGAGGUUCCCGCUACGAGAACCAUCCUCGUAGCUUCG
GUGGUGCAGAUGAACUUCAGGGUCA
F:
(SEQ ID NO: 26)
5′-GGGAACCGAUGGUUGGUUCCCGCUACGAGAGUGGACCUCGUAGCUUC
GGUGGUGCAGAUGAACUUCAGGGUCA

RNA molecules were purchased (from Integrated DNA Technologies, Inc., for short RNAs, e.g., siRNAs and/or DsiRNAs) or prepared by transcription of PCR amplified DNA templates; synthetic DNA molecules coding for the sequence of the designed RNA were purchased already amplified by PCR using primers containing the T7 RNA polymerase promoter (see PCT/US2013/058492, filed Sep. 6, 2013, incorporated by reference in its entirety herein). PCR products were purified using the QiaQuick PCR purification kit and RNA molecules were prepared enzymatically by in vitro transcription using T7 RNA polymerase. For the visualization of assembled RNA NPs quality control experiments, [³²P]Cp labeled RNA molecules were used (T4 RNA ligase is used to label the 3′-ends of RNA molecules by attaching [³²P]Cp¹⁹). In the case of the initial radiolabel native-PAGE assays, radiolabeled RNA scaffold strand was mixed with concatenated strands individually followed by the assembly protocol⁴. For dicing functional control experiments, RNA molecules were co-transcriptionally a[P³²]-ATP body-labeled. Native PAGE experiments were performed as described²⁰. Typically, assembly experiments reported were analyzed at 10° C. on 7% (29:1) native polyacrylamide gels in the presence of 89 mM Tris-borate, pH 8.3, 2 mM Mg(OAc)₂. A Hitachi FMBIO II Multi-View Imager was used to visualize SYBR Gold stained R/DNA hybrids.

RNA/DNA Nanoparticles and Hybrid Assemblies and Native PAGE.

The designing principles and production of RNA strands entering the composition of the nanorings functionalized with six siRNAs is comprehensively described elsewhere (Afonin et al Multifunctional RNA nanoparticles. Nano letters 2014, 14, 5662-5671). RNA-DNA hybrids were assembled as detailed elsewhere (Afonin et al, Activation of different split functionalities on re-association of RNA-DNA hybrids. Nature nanotechnology 2013, 8, 296¬304) and RNA/DNA nanorings were produced co-transcriptionally (Afonin et al, Co-transcriptional assembly of chemically modified RNA nanoparticles functionalized with siRNAs. Nano letters 2012, 12, 5192-5195). Briefly, synthetic DNA molecules coding for the sequence of the designed RNA were purchased and amplified by PCR using primers containing the T7 RNA polymerase promoter. Corresponding DNA templates were mixed together with ssDNA required for hybrid formations and RNA-DNA nanoparticles were produced enzymatically by in vitro run-off transcription using wildtype T7 RNA polymerase. Transcription was performed in 80 mM HEPES-KOH, pH 7.5; 2 mM spermidine; 50 mM DTT; 25 mM MgCl₂; 1 mM NTPs; 0.2 μM of DNA templates, 50 μM of ssDNA for hybrids; and T7 RNA polymerase. The resulting co-transcriptionally assembled nanorings functionalized with six hybrids were purified by native-PAGE and further analyzed. Typically, all assembly experiments were analyzed at 10° C. on 7% (37.5:1) native polyacrylamide gels in the presence of 89 mM Tris-borate, pH 8.3 and 2 mM Mg(OAc)₂. ChemiDoc MP System was used to visualize fluorescently labeled (Alexa 488 and Alexa 546) and EtBr stained RNA-, RNA-DNA- and DNA-RNA-based assemblies.

### FRET Studies.

Re-association of cognate RNA-DNA hybrids in vitro was tracked with FRET measurements using a FluoroMax3 (Jobin-Yvon, Horiba). All fluorescently labeled RNAs are presented in Supporting Information (Fluorescently labeled molecules). For all the experiments, the excitation wavelength was set at 460 nm, and the excitation and emission slit widths were set at 2 nm. Alexa 488 labeled constructs were first incubated for two minutes at 37° C., and then Alexa 546 labeled constructs were added. Upon excitation at 460 nm, the emissions at 520 nm and 570 nm were recorded simultaneously every 30 seconds to follow the process of re-association through FRET measurements. This experiment was also carried out with hybrids individually pre-incubated with L2K (Invitrogen) in the amounts relevant for the transfection conditions. In vitro fluorescent experiments show no re-association of these complexes in solution thus, the re-association occurs only in cells.

### Transfection of Human Breast Cancer Cells MDA-MB-231.

MDA-MB-231 cells either with or without eGFP were grown in a 5% CO₂ incubator in DMEM (Gibco BRL) supplemented with 10% FBS and penicillin-streptomycin. All transfections in this project were performed using L2K. 50× solutions of hybrids were individually pre-incubated at room temperature with L2K for 30 minutes. RNA-DNA nanorings were co-transfected with their cognate RNA-DNA hybrids (at six-fold higher concentrations). To avoid re-association in media, cognate RNA-DNA hybrids were pre-incubated with L2K separately. Prior to each transfection, the cell media was swapped with OPTI-MEM mixed with prepared 50× of hybrids/L2K complexes to the final concentration of 1×. The cells were incubated for 4 hours followed by the media change (D-MEM, 10% FBS, 1% pen-strep) (Afonin wt al, Triggering of RNA Interference with RNA-RNA, RNA-DNA, and DNA-RNA Nanoparticles. ACS nano 2015, 9, 251-259 and Afonin et al, Multifunctional RNA nanoparticles. Nano letters 2014, 14, 5662-5671 and Afonin et al, Activation of different split functionalities on re-association of RNA-DNA hybrids. Nature nanotechnology 2013, 8, 296-304).

### Confocal Microscopy.

The intracellular re-association of RNA-DNA hybrids was assessed through FRET Afonin wt al, Triggering of RNA Interference with RNA-RNA, RNA-DNA, and DNA-RNA Nanoparticles. ACS nano 2015, 9, 251-259 and Afonin et al, Multifunctional RNA nanoparticles. Nano letters 2014, 14, 5662-5671 and Afonin et al, Activation of different split functionalities on re-association of RNA-DNA hybrids. Nature nanotechnology 2013, 8, 296¬304). All measurements were performed using a LSM 710 confocal microscope (Carl Zeiss) with a 63×, 1.4 NA magnification lens. All images were taken with a pinhole adjusted to 1 airy unit. Fluorescently labeled hybrid nanoparticles and cognate hybrids were individually pre-incubated with L2K and co-transfected into cells. On the next day, the samples were fixed by incubation in 4% paraformaldehyde for 20 minutes at room temperature. Images of the cells were then taken to assess the appearance of FRET within the sample. For Alexa 488 imaging, the 488 nm line of an Argon laser was used as excitation and the emission was collected between 493 and 557 nm. For Alexa 546 imaging, a DPSS 561 laser was used for excitation, and emission was collected between 566 and 680 nm. In order to evaluate the sensitized emission through FRET, images were taken exciting the sample with the 488 nm line and collecting emission between 566 and 680 nm. Because of spectral overlap, the FRET signal is contaminated by donor emission into the acceptor channel and by the excitation of acceptor molecules by the donor excitation wavelength. This bleed-through was assessed via measurements performed with samples transfected with individual dyes and mathematically removed from the images of FRET.

### Flow Cytometry Experiments.

MDA-MB-231 cells expressing GFP were grown in 12-well plates (1.0×10⁵ cells per well) and lifted with a cell dissociation buffer (Gibco BRL). The level of GFP was measured by fluorescence-activated cell sorting (FACS) analysis on a FACScalibur flow cytometer (BD Bioscience) (Baugh et al, Journal of molecular biology 2000, 301, 117-128; Gupta et al, Journal of controlled release: official journal of the Controlled Release Society 2015, 213, 142-151; Gupta et al, Nanomedicine 2015, 1-14).

At least 20,000 events were collected and analyzed using the CellQuest software. For statistical analysis, the geometric mean fluorescence intensity (gMFI) and standard error of the mean (SEM) were calculated and plotted (Afonin et al, Triggering of RNA Interference with RNA-RNA, RNA-DNA, and DNA-RNA Nanoparticles. ACS nano 2015, 9, 251-259 and Afonin et al, Multifunctional RNA nanoparticles. Nano letters 2014, 14, 5662-5671).

### HIV-1 Experiments.

HeLa cells were cultured in a 24-well plate, in a 5% CO₂ incubator, in antibiotic-free DMEM (Gibco BRL) supplemented with 5% FBS (Atlanta biologicals). Nucleic acids and L2K were mixed an concentrations higher than recommended by the manufacturer; for each well, 0.66 jtg HIV-1 infectious clone pNL4-3 (Adachi et al, Journal of virology 1986, 59, 284-291), 0.05 jtg gaussia luciferase reporter plasmid pGLuc (NEB) and either 2.5 or 10 μmol of RNA/DNA hybrids, were combined in 15 jtl Optimem. 2.4 jtl L2K was mixed with 15 jtl Optimem and incubated for 5 minutes before combining with the nucleic acids. To avoid re-association in media, cognate RNA-DNA hybrids were pre-incubated with L2K separately. The transfection mix was incubated for 30 minutes at room temperature then added dropwise to cells. After incubation for 8 hours, the media were removed and replaced with DMEM supplemented with 5% FBS, 100 U/ml penicillin, 100 jtg/ml streptomycin, and 2 mM L-glutamine (Gibco BRL). After 48 hours, supernatant was collected and supplemented with 0.5% Triton X-100 to inactivate infectious HIV-1 particles. Samples of supernatant were assayed for gaussia luciferase activity, as a reporter for transfection efficiency and toxicity, using Biolux (NEB) following the manufacturer's instructions; and for reverse transcriptase (RT), a component of the HIV-1 virion, as described previously⁴⁸. Virus release was calculated as the ratio of RT to luciferase signal. Cells were also harvested in 2x Laemmli buffer (20% glycerol, 4% SDS, 0.01% bromophenol blue, 700 mM β-mercaptoethanol, 100 mM Tris pH 6.8) and probed by western blotting with HIV immunoglobulin (NIH AIDS Reagent Program Catalog #3957) and monoclonal mouse anti-GAPDH (clone 6C5—Santa Cruz), and species-specific horseradish peroxidase-conjugated secondary antibodies (Thermo Scientific). Bands were revealed using chemiluminescence and the signal was recorded using a Chemidoc XRS+ system (Biorad). Band volumes were determined using Image Lab software (Biorad); to determine Gag expression, volumes for p55, p41 and CA were summed, and then divided by the corresponding GAPDH volume.

### In Silico Predictions of RNA-DNA Hybrid Re-Association.

In the novel computational approach (, an estimated free energy of a large variety of secondary structure states of the four involved sense and antisense RNA and DNA strands is computed. The free energy contributions of base pairing are based on base pair stacking energies for RNA/RNA, DNA/DNA and RNA/DNA interactions. Entropy changes with respect to the unfolded state are estimated using a geometric distance-based model. In this approach, for each pair of nucleotides, maximally attainable residue-residue distances are computed for each examined secondary structure state. Each formed based pair can lead to a reduction of these maximally extended conformations and thus to a reduction in entropy. This distance-based approach is a general model for estimating entropy contributions that is not limited by the number of involved nucleotide strands and is applicable to pseudoknotted structures (Afonin et al, Computational and Experimental Studies of Reassociating RNA/DNA Hybrids Containing Split Functionalities. Methods in enzymology 2015, 553, 313-334). From this list of states, concentrations of the RNA/DNA hybrids, the re-associated RNA and DNA duplexes as well as a variety of intermediate states can be estimated.

The computational approach also identifies pairs of secondary structures that have a different strand connectivity and differ by only one additionally placed helix. The minimum energetic cost to place one additional helix to transition from one inter-strand connectivity to a different inter-strand connectivity is used to estimate the free energy of activation. For the case of RNA/DNA hybrids it was observed that the estimated free energy of activation (going from the non-re-associated state to an intermediate complex consisting of four strands) can lead to a qualitative prediction where the RNA/DNA hybrids can readily re-associate but are kinetically trapped due to short toehold lengths (leading to a high free energy of activation of the transition state).

The free energy of activation from the state of RNA/DNA hybrid duplexes to the quadruplex intermediate state is estimated by estimating the free energy change of the formation of the first duplex between the cognate toehold regions (without unfolding of any bases pairs of the RNA/DNA hybrid start state). In other words, for an RNA/DNA hybrid structure with 6nt toeholds, the free energy change of forming a 6nt helix between the cognate toehold regions of the non-reasssociated RNA/DNA hybrids strands is estimated as shown in FIG. 4B for different toehold lengths and strand concentrations. Other strand configurations (such as complexes involving 3 strands) were ruled out as important transition states because they are predicted to have unfavorable free energies.

The algorithm and computational method is also used to predict RNA secondary structures. A complete enumeration of all secondary structures is not feasible for longer nucleotide sequences because of a combinatorial explosion of possible secondary structures. Because of that, the algorithm performs an enumeration of secondary structures that are generated by choosing helices from a set of “core helices”. Those “core helices” are defined as helices that consist of at least 6 base pairs and that cannot be extended further on either end by Watson-Crick or GU base pairs.

### Representation of Secondary Structures.

Each secondary structure is internally represented by a matrix of maximum and minimum distances between nucleotides. For nucleotide pairs of single-stranded regions of the same strand, their maximum distance is computed as the product of the number of separating nucleotides times a maximum distance per nucleotide. If both nucleotides are part of the same base pair, their maximum and minimum distances are set to a constant value. If two nucleotides belong to different nucleotide strands without inter-strand base pairing, their maximum distance is given through the inverse of the strand concentrations. For each folded base pair, all maximum and minimum nucleotide-nucleotide distances are updated to fulfill these rules. The maximum and minimum distances between residues define an estimated volume of conformational freedom that is used to compute the conformational entropy of a secondary structure. Importantly, the approach provides estimated entropy contributions for potentially pseudoknotted structures consisting of multiple strands. Free energy contributions of nucleotide base pairing are computed using base pair stacking parameters for RNA/RNA, DNA/DNA and RNA/DNA interactions (Sugimoto et al, Biochemistry 1995, 34, 11211; Mathews et al, JMolBiol 1999, 288, 911; SantaLucia et al, Proc NatlAcad Sci USA 1998, 95, 1460). Because this matrix data structure in “distance space” is memory-intensive, another more compact representation is utilized in form of a vector of integer numbers that indicate which of the core helices are part of the structure. A “0” at position i in that integer vector indicates that core helix i is not part of the structure; a “1” at position i indicates that a core helix i is in its full length present in the secondary structure. Indices greater than one describe different partially placed variants of a helix. The matrix representation of secondary structures facilitates entropy estimations, while the integer vector representation is memory-efficient. The program is able to inter-convert between the distance matrix representation and the integer vector representations thus making the distance matrix available only when needed.

### Algorithm.

Briefly, this folding algorithm works as follows: Two priority queues A and B for storing free energy-ranked secondary structures are generated. To prevent the combinatorial explosion of the computational search, the implementation of the priority queue data structure is such that it automatically deletes the lowest-priority elements, if the number of stored elements exceeds a specified maximum size (this maximum size is set to 1000 elements). The two priority queues hold the integer vector representations of the partially folded nucleic acid complexes such that the contained structures are at any given time sorted by the estimated free energy of folding. The algorithm starts by generating an array of “core helices” (defined above) that is sorted in order of free energy. In addition, a second array of non-core “short helices” with lengths from 3 to 5 base pairs is generated. Next, the integer vector representation of an unfolded structure (containing zero base pairs) is added to priority queue A. If there are n core helices, the search consists of n rounds. In the first round, the top-ranking element of queue A (which happens to be the previously added unfolded structure) is removed from queue A. Four variants of that structure are generated and added to queue B. These four variants are: i) core helix 1 being unplaced, ii) core helix 1 fully placed, iii) and iv) core helix 1 placed to half its length from either end. Helix candidates can be shortened in order to not conflict with already placed helices. Structures in which a helix cannot be placed are not stored. In other words, in round 1 the decision is made to what extent core helix 1 is placed. For each structure composed of a combination of core helices, another structural variant is generated by placing the previously generated short helices in the order of free energy, leading for the first round to up to 4×2=8 stored structural variants. In the first round, queue B has the role of a “receiving” queue, and queue A has the role of “producing” queue. In the second round, queue A becomes the receiving queue and queue B the producing queue. For a general round i, and for each structure removed from the producing queue, the different variants with respect to the placement of core helix i are generated and the resulting secondary structures are added to the receiving queue (one version without additional small helices and one version with additional small helices). This process is continued until the producing queue is empty and all core helices have been explored.

In this fashion, all combinations of core helices are searched quasi-exhaustively while placing additional small helices in order of free energy for each examined combination of core helices. Helices can be pseudoknotted with the constraint that each helix can be non-nested with respect to at most one other helix. This approach thus combines entropy estimations and a tunable search strategy that avoids redundant structure evaluations and is thus applicable (and used) for estimating the partition function.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### REFERENCES

The following specific references, also incorporated by reference, are indicated above by corresponding reference number.
1. Li, N. et al. Technical and biological issues relevant to cell typing with aptamers.

J Proteome Res 8, 2438-2448 (2009).
2. Li, N., Larson, T., Nguyen, H.H., Sokolov, K.V. & Ellington, A.D. Directed evolution of gold nanoparticle delivery to cells. Chemical communications (Cambridge, England) 46, 392-394(2010).
3. Li, N., Nguyen, H.H., Byrom, M. & Ellington, A.D. Inhibition of cell proliferation by an anti-EGFR aptamer. PloS one 6, e20299 (2011).
4. Afonin, K. A. et al. Design and self-assembly of siRNA-functionalized RNA nanoparticles for use in automated nanomedicine. Nat Protoc 6, 2022-2034 (2011).
5. Grabow, W. W. et al. Self-assembling RNA nanorings based on RNAI/II inverse kissing complexes. Nano Lett 11, 878-887 (2011).
6. Rose, S. D. et al. Functional polarity is introduced by Dicer processing of short substrate RNAs. Nucleic Acids Res 33, 4140-4156 (2005).
7. Afonin, K.A., Danilov, E.O., Novikova, I.V. & Leontis, N.B. TokenRNA: a new type of sequence-specific, label-free fluorescent biosensor for folded RNA molecules. Chembiochem 9, 1902-1905 (2008).
8. Grate, D. & Wilson, C. Laser-mediated, site-specific inactivation of RNA transcripts.

Proc Natl Acad Sci USA 96, 6131-6136 (1999).
9. Kolpashchikov, D.M. Binary malachite green aptamer for fluorescent detection of nucleic acids. J Am Chem Soc 127, 12442-12443 (2005).
10. Stojanovic, M.N. & Kolpashchikov, D.M. Modular aptameric sensors. J Am Chem Soc 126, 9266-9270 (2004).
11. Afonin, K. A. et al. In vitro assembly of cubic RNA-based scaffolds designed in silico. Nat Nanotechnol 5, 676-682 (2010).
12. Afonin, K. A. et al. Activation of different split functionalities on re-association of RNA-DNA hybrids. Nat Nanotechnol 8, 296-304 (2013).
13. Shu, D., Shu, Y., Haque, F., Abdelmawla, S. & Guo, P. Thermodynamically stable RNA three-way junction for constructing multifunctional nanoparticles for delivery of therapeutics.

Nat Nanotechnol 6, 658-667 (2011).
14. Chang, C. I. et al. Enhanced intracellular delivery and multi-target gene silencing triggered by tripodal RNA structures. J Gene Med 14, 138-146 (2012).
15. Lee, Y. S. et al. Silencing by small RNAs is linked to endosomal trafficking. Nat Cell Biol 11, 1150-1156 (2009).
16. Sen, G. L. & Blau, H. M. Argonaute 2/RISC resides in sites of mammalian mRNA decay known as cytoplasmic bodies. Nat Cell Biol 7, 633-636 (2005).
17. Nakashima, Y., Abe, H., Abe, N., Aikawa, K. & Ito, Y. Branched RNA nanostructures for RNA interference. Chemical communications (Cambridge, England) (2011).
18. Zhang, H., Kolb, F. A., Brondani, V., Billy, E. & Filipowicz, W. Human Dicer preferentially cleaves dsRNAs at their termini without a requirement for ATP. Embo J 21, 5875-5885 (2002).
19. Afonin, K. A., Cieply, D. J. & Leontis, N. B. Specific RNA self-assembly with minimal paranemic motifs. J Am Chem Soc 130, 93-102 (2008).
20. Afonin, K. A. & Leontis, N. B. Generating new specific RNA interaction interfaces using C-loops. J Am Chem Soc 128, 16131-16137 (2006).
21. Tang, G. et al. EMAN2: an extensible image processing suite for electron microscopy.

Journal of structural biology 157, 38-46 (2007).
22. Wakabayashi, H., Varfaj, F., Deangelis, J. & Fay, P. J. Generation of enhanced stability factor VIII variants by replacement of charged residues at the A2 domain interface. Blood 112, 2761-2769 (2008).
23. Kim, T. et al. In Silico, In Vitro, and In Vivo Studies Indicate the Potential Use of Bolaamphiphiles for Therapeutic siRNAs Delivery. Mol Ther Nucleic Acids 2, e80 (2013).
24. Afonin, K. A. et al. Design and self-assembly of siRNA-functionalized RNA nanoparticles for use in automated nanomedicine. Nat Protoc 6, 2022-2034 (2011).
25. Rose, S. D. et al. Functional polarity is introduced by Dicer processing of short substrate RNAs. Nucleic acids research 33, 4140-4156 (2005).
26. Afonin, K. A., Danilov, E. O., Novikova, I. V. & Leontis, N. B. TokenRNA: a new type of sequence-specific, label-free fluorescent biosensor for folded RNA molecules. Chembiochem 9, 1902-1905 (2008).
27. Grate, D. & Wilson, C. Laser-mediated, site-specific inactivation of RNA transcripts. Proceedings of the National Academy of Sciences of the United States of America 96, 6131-6136 (1999).
28. Li, N. et al. Technical and biological issues relevant to cell typing with aptamers. Journal of proteome research 8, 2438-2448 (2009).
29. Low, J. T. et al. SHAPE-directed discovery of potent shRNA inhibitors of HIV-1. Mol Ther 20, 820-828 (2012).
30. Afonin, K. A. et al. Activation of different split functionalities on re-association of RNA-DNA hybrids. Nat Nanotechnol 8, 296-304.
31. Shapiro B et al. Protocols for the In silico Design of RNA Nanostructures. In: Nanostructure Design Methods and Protocols. Totowa, N. J.: Humana Press; 2008. p. 93-115 [Book Chapter]
32. Bindewald et al. Computational strategies for the automated design of RNA nanoscale structures from building blocks using NanoTiler. Mol. Graph. Model. 27(3): 299-308, 2008.
33. Shapiro B A et al. Protocols for the in silico design of RNA nanostructures.

Methods Mol. Biol. 474: 93-115, 2008.
34. Martinez H M et al. RNA2D3D: A program for Generating, Viewing, and Comparing 3-Dimensional Models of RNA. J. Biomol. Struct. Dyn. 25: 669-83, 2008. [Journal]
35. Bindewald E et al. RNAJunction: a database of RNA junctions and kissing loops for three-dimensional structural analysis and nanodesign. Nucleic Acids Res. 36: D392-7, 2008.
36. Yingling Y G et al. Computational Design of an RNA Hexagonal Nanoring and an RNA Nanotube. Nano Lett. 7(8): 2328-2334, 2007. Full Text Article.
37. Hastings W et al. Structural and dynamical classification of RNA single-base bulges for nanostructure design. J. Comp. Theor. Nanoscience. 3: 63-77, 2006.
38. Paliy M et al. Molecular dynamics study of the RNA ring nanostructure: a phenomenon of self-stabilization. Phys Biol. 6(4): 46003, 2009.

1-25. (canceled)

26. A method of triggering one or more functionalities in a cell comprising:
(a) administering an effective amount of a first inactive nanoparticle comprising a first set of DNA and/or RNA strands and a first ssRNA toehold;
(b) administering an effective amount of a second inactive nanoparticle comprising a second set of DNA and/or RNA strands and a second ssRNA toehold;
wherein the strands of the first inactive nanoparticle are the reverse complements of the strands of the second inactive nanoparticle,
wherein the first and second inactive nanoparticles are capable of undergoing reassociation of their strands to produce one or more functionalities, and
wherein the reassociation of strands of the first and second nanoparticles is triggered by the interaction of the first and second ssRNA toeholds.

27. The method of triggering one or more functionalities of claim 26, wherein the one or more functionalities is selected from the group consisting of transcription, aptamer, optical response, and Dicer substrate.

28. The method of triggering one or more functionalities of claim 26, wherein the ssRNA toeholds are 2, 4, 6, 8, 10, or 12 nucleotides in length.

29. The method of triggering one or more functionalities of claim 26, wherein the ssRNA toeholds are at least 4 nucleotides in length.

30. The method of triggering one or more functionalities of claim 26, wherein the ssRNA toeholds impart greater stability, smaller size, and greater production yields by run-off transcription as compared to nanoparticles with ssDNA toeholds.

31. The method of triggering one or more functionalities of claim 26, wherein the first or second nanoparticle is a nanoring, nanotube, or nanocube comprising one or more hybrid duplex arms comprising the first or second ssRNA toehold.

32. The method of triggering one or more functionalities of claim 26, wherein the first or second nanoparticle is an RNA/DNA duplex comprising the first or second ssRNA toehold.

33. The method of triggering one or more functionalities of claim 27, wherein the one or more functionalities is a Dicer substrate, further wherein the Dicer substrate inhibits a target gene.

34. A method of inhibiting a target gene in a cell comprising:
(a) administering an effective amount of a first inactive nanoparticle comprising a first set of DNA and/or RNA strands and a first ssRNA toehold;
(b) administering an effective amount of a second inactive nanoparticle comprising a second set of DNA and/or RNA strands and a second ssRNA toehold;
wherein the strands of the first inactive nanoparticle are the reverse complements of the strands of the second inactive nanoparticle,
wherein the first and second inactive nanoparticles are capable of undergoing reassociation of their strands to produce one or more functionalities which inhibit a target gene in the cell, and
wherein the reassociation of strands of the first and second nanoparticles is triggered by the interaction of the first and second ssRNA toeholds.

35. The method of claim 34, wherein the one or more functionalities is selected from the group consisting of transcription, aptamer, optical response, and Dicer substrate.

36. The method of claim 34, wherein the ssRNA toeholds are 2, 4, 6, 8, 10, or 12 nucleotides in length.

37. The method of claim 34, wherein the ssRNA toeholds are at least 4 nucleotides in length.

38. The method of claim 34, wherein the ssRNA toeholds impart greater stability, smaller size, and greater production yields by run-off transcription as compared to nanoparticles with ssDNA toeholds.

39. The method of claim 34, wherein the first or second nanoparticle is a nanoring, nanotube, or nanocube comprising one or more hybrid duplex arms comprising the first or second ssRNA toehold.

40. The method of claim 34, wherein the first or second nanoparticle is an RNA/DNA duplex comprising the first or second ssRNA toehold.

41. The method of claim 35, wherein the one or more functionalities is a Dicer substrate, further wherein the Dicer substrate inhibits the target gene.

42. A method of triggering one or more functionalities in a cell comprising administering a therapeutically effective amount of an activatable nanoparticle system comprising one or more split functionalities comprising a first inactive nanoparticle comprising a first set of DNA and/or RNA strands and a first ssRNA toehold and a second inactive nanoparticle comprising a second set of DNA and/or RNA strands and a second ssRNA toehold, wherein the first ssRNA toehold comprises the reverse complement of the second ssRNA toehold, wherein the first ssRNA toehold and the second ssRNA toehold are each 2 to 8 nucleotides in length, wherein the first and second ssRNA toeholds associate to provide for the activation of the nanoparticle system.

43. The method of claim 42, wherein the one or more split functionalities is selected from the group consisting of split transcription, split aptamer, split optical response, and split Dicer substrate.

44. The method of claim 42, wherein the ssRNA toeholds are 4 to 8 nucleotides in length.

45. The method of claim 42, wherein the ssRNA toeholds impart greater stability, smaller size, and greater production yields by run-off transcription as compared to nanoparticles with ssDNA toeholds.
